SEQUENCE LISTING (17 sequences)

SEQ ID NO: 1 - DNA, 1797 bp, Macaca mulatta
atgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccggttcggtttactacaagccctcctcgcccccgacgcccaccaccccgggcttccaggtgcagcacagccccatgtgggacgacccggggtctctccacaacttccaccagaactacgtggccactacgcacatgatcgagcagaggaaaactccagtttcccgcctctccctcttctcctttaagcaatcgccccctggcaccccggtgtctagttgccagatgcgcttcgacgggcccctgcacgtccccatgaacccggagcccgccagcagccaccacgtggtggacgggcagacctttgctgtacccaaccccattcgcaagcccgcgtccatgggcttcccgggcctgcagatcggccacgcgtcgcagctgcttgacacgcaggtgccctcgccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatatgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcagatgagtggagatgacacccagcatatccagcaattctatgatctcctgactggctccatggagatcatccggggctgggcggagaagatccctggcttcgcagacctgcccaaagccgaccaggacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagaggcacgggctcaaggaacccaagagagtggaagaactgcaaaacaagattgtaaattgtctcaaagaccatgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaa

SEQ ID NO: 2 - protein, 598 aa, Macaca mulatta
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPASSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 3 - DNA, 1797 bp, Homo sapien
atgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaa

SEQ ID NO: 4 - DNA, 21 bp, Artificial Sequence
  Palindromic sequence in promoter for pro-opiomelanocortin gene
tgacctttnnnnnaaaggtca

SEQ ID NO: 5 - DNA, 17 bp, Artificial Sequence
  Sequence recognized by recognized by the NURR1-RXR heterodimer
aggtcannnaaaggtca

SEQ ID NO: 6 - DNA, 8 bp, Artificial Sequence
  NGFI-B response element or binding site
aaaggtca

SEQ ID NO: 7 - DNA, 15 bp, Artificial Sequence
  PCR primer forward
atgccttgtgttcag

SEQ ID NO: 8 - DNA, 18 bp, Artificial Sequence
  PCR primer reverse
ttagaaaggtaaagtgtc

SEQ ID NO: 9 - DNA, 26 bp, Artificial Sequence
  PCR primer forward
atgccttgtgttcaggcgcagtatgg

SEQ ID NO: 10 - DNA, 33 bp, Artificial Sequence
  PCR primer reverse
ttagaaaggtaaagtgtccaggaaaagtttgtc

SEQ ID NO: 11 - DNA, 9 bp, Artificial Sequence
  Oligonucleotide containing an XhoI restriction enzyme site
gctcgagaa

SEQ ID NO: 12 - DNA, 18 bp, Artificial Sequence
  PCR primer forward
gggtcctcgcctcaagga

SEQ ID NO: 13 - DNA, 20 bp, Artificial Sequence
  PCR primer reverse
cggagctgtattctcccgaa

SEQ ID NO: 14 - DNA, 26 bp, Artificial Sequence
  Probe
ccagccccgcttctcagagctacagt

SEQ ID NO: 15 - DNA, 16 bp, Artificial Sequence
  PCR primer forward
gctggaccaaacacaa

SEQ ID NO: 16 - DNA, 20 bp, Artificial Sequence
  PCR primer reverse
gccatccagccactcagtct

SEQ ID NO: 17 - DNA, 28 bp, Artificial Sequence
  Probe
tggttcccagttttttatctgcactgcc

(19) United States
(12) Patent Application Publication
(10) Pub. No.: US 2006/0040298 A1
(43) Pub. Date: Feb. 23, 2006

(54) Rhesus monkey NURR1 nuclear receptor

(76) Inventors: Azriel Schmidt, Bryn Mawr, PA (US); Le Thi Duong, Lansdale, PA (US); Maureen Pickarski, Ambler, PA (US)

Correspondence Address: MERCK AND CO., INC, P O BOX 2000, RAHWAY, NJ, 07065-0907, US

(21) Appl. No.: 11/198,640

(22) Filed: Aug. 5, 2005

Related U.S. Application Data
(60) Provisional application No. 60/599,279, filed on Aug. 5, 2004.

(51) Int. Cl. C12Q 1/68; G01N 33/53; C07H 21/04; C12P 21/06; C12N 5/06; C07K 14/72; C07K 16/28

(52) U.S. Cl. 435/6; 435/69.1; 435/7.1; 435/320.1; 435/363; 530/350; 536/23.5; 530/388.22

(57) ABSTRACT

The Macaca mulatta (rhesus monkey) NURR1 nuclear receptor and the nucleic acid encoding the rhesus monkey NURR1 are described. Further described are methods for identifying analytes which modulate expression or activity of the rhNURR1 for treating or inhibiting inflammatory diseases such as osteoarthritis, bone disorders, neurological such as Parkinson's disease, psychotic diseases such as bipolar diseases, and prostate disorders.

### CROSS REFERENCE TO RELATED APPLICATIONS

This application claims the benefit of U.S. Provisional Application No. 60/599,279, filed Aug. 5, 2004, the contents of which are incorporated herein by reference in their entirety.

### BACKGROUND OF THE INVENTION

(1) Field of the Invention

The present invention relates to the Macaca mulatta (rhesus monkey) NURR1 nuclear receptor and the nucleic acid encoding the rhesus monkey NURR1. The present invention further relates to methods for identifying analytes which modulate expression or activity of NURR1 for treating or inhibiting inflammatory diseases such as osteoarthritis, bone disorders, neurological and psychotic disorders, and prostate disorders.

(2) Description of Related Art

NURR1 (nuclear receptor related 1) is a transcription factor that belongs to the superfamily of nuclear receptors. The nuclear receptor superfamily includes receptors for various steroid hormones, retinoids, thyroid hormone, and 15 vitamin D. The superfamily further includes a large group of receptors classified as orphan receptors because their natural ligands are not yet known. NURR1 is currently classified as an orphan receptor. NURR1 has been variously known as regenerating liver nuclear receptor 1 (RNR-1), nuclear receptor subfamily 4, group A, member 2 (NR4A2), HZF-3, TINUR, or T-cell nuclear receptor (NOT, NOT1, or NOT1A).

NURR1 exhibits a close structural relationship to the orphan receptors NUR77 (also referred to as NGFIB/N10/NAK) (Hazel et al., Proc. Natl. Acad. Sci. USA. 85: 8444-8448 (1988); Milbrant, Neuron 1: 183-188 (1998); Ryseck et al., EMBO J. 8: 3327-3335 (1989); Nakai et al., Mol. Endocrinol. 4: 1438-1443 (1990)) and NOR-1 (also called MINOR/TEC) (Ohkura et al., Biochem. Biophys. Res. Comm. 205: 1959-1965 (1994); Maruyama et al., Cancer Lett. 96:117-122 (1995); Hedvat and Irving, Mol. Endocrinol. 9: 1692-1700 (1995)). All three of these orphan nuclear receptors comprise the NURR subfamily of nuclear receptors which has been characterized as being able to bind to the same cis-acting consensus nucleotide sequence—the NGF1-B response element (NBRE)—to regulate target gene expression (Ohkura et al., Biochem. Biophys. Res. Comm. 205: 1959-1965 (1994); Wilson et al., Science 252: 1296-1300 (1991); Murphy et al., Gene Express. 5: 169-179 (1995)). For example, NURR1 and NUR77 regulate expression of the Corticotropin Releasing Hormone (CRH) and proopiomelanocortin (POMC) genes by interacting with specific cis-acting sequences in their proximal promoter region.

Unlike most nuclear receptors, the NURR subfamily of nuclear receptors are encoded by immediate early genes whose expression can be differentially induced in response to a variety of extracellular stimuli such as growth factors (Hazel et al., ibid.; Milbrandt, ibid.), neurotransmitters (Watson and Milbrandt, Mol. Cell. Biol. 9: 42134219 (1989)), and polypeptide hormones (Wilson et al., Mol. Cell. Biol. 13: 861-868 (1993); Murphy and Conneely, ibid.; Davis and Lau, Mol. Cell Biol. 14: 3469-3483 (1994)). NURR1 and NUR77 are rapidly induced by CRH in primary pituitary cells, resulting in increased synthesis of POMC (Murphy and Conneely, ibid.). Glucocorticoid repression of the POMC gene is mediated by glucocorticoid receptor dependent inhibition of activation of the POMC gene by NURR1 and NUR77 (Evans, ibid.; Philips et al., Mol. Cell. Biol. 17: 5952-5959 (1997)). Because NOR-1 possesses an identical DNA binding domain and is capable of binding the same cis-acting consensus sequence as NURR1 and NU77, it has been included in the NURR subfamily. Therefore, the close structural relationship, the identical cis-acting consensus sequence, and the ability of the different members of the NURR subfamily of transcription factors to functionally complement one another are strong indications that the NURR subfamily members might have redundancy of function.

Like most nuclear receptors of the nuclear receptor superfamily, NURR1 consists of an amino-terminal transactivation function 1 (AF1) near the amino terminus, which enables ligand-independent transcription activation; a core DNA binding domain (DBD) located near the center of the protein, which contains two highly conserved zinc finger motifs and which binds to specific nucleotide sequences; a hinge region, which permits protein flexibility to allow for simultaneous receptor dimerization and DNA binding; a conserved ligand binding domain (LBD) near the carboxy terminus, which includes a dimerization interface; and, a carboxyl-terminal activation function 2 (AF2) near the carboxy terminus, which enables ligand-independent transcription activation. Many nuclear receptors act as dimers, either as homodimers or as heterodimers. The dimerization interface in the LBD, the I-box, has been mapped to a region in the carboxyl terminal part of the LBD that corresponds to helix 10 in the canonical nuclear receptor LBD structure (Perlmann et al., Mol. Endocrinol. 10: 958-966 (1996); Lee et al. Mol. Endocrinol. 12: 325-332 (1998)). The I-box is well conserved among several dimerizing receptors, including NURR1 and NOR 1. In NURR1, the 1-box is the region at amino acids 524-556 of the full length protein. NURR1 is also able to bind to DNA as a monomer (Wilson et al., Science 252: 1296 (1991)).

NURR1 forms heterodimers with retinoid X receptor (RXR) and, in contrast to most other RXR heterodimers, this dimer can be activated by retinoids (Perlmann and Jansson, Genes Dev. 9: 769-782 (1995); Forman et al. (1995); Zetterstrom et al., Science 276: 248-250 (1996)). U.S. Pat. No. 6,458,926 to Evans et al. also discloses that RXR can interact productively with NURR1, which in the absence of RXR, is capable of binding DNA as a monomer. As a result of this interaction with RXR, the constitutive activity of NURR1 is suppressed, and the resulting complex becomes responsive to RXR-selective ligands (for example, 9-cis retinoic acid). The unique ability of the NURR1/RXR heterodimer complex to transduce RXR signals establishes a novel response pathway. The patent suggests that heterodimer formation imparts allosteric changes upon the LBD of the NURR1. These allosteric changes confer transcriptional activities onto the NURR1-RXR heterodimer that are distinct from those of the NURR1 or RXR monomers. This permits a limited number of regulatory proteins to generate a diverse set of transcriptional responses to multiple hormonal signals. Mutations in the 1-box of NURR1 have been shown to inhibit dimerization with RXR while maintaining its monomeric transcriptional activity (Published US Patent Application No. 2002059303 to Perlman and Aarnisalo).

While NURR1 has been classified as an orphan receptor because the natural ligand for NURR1 has not been identified, Wang et al. (Nature 423: 555-560 (2003)) provide crystallographic and transcriptional assay data that suggests NURR1 might be a ligand-independent nuclear receptor. Their crystallographic analysis of NURR1 showed that while the NURR1 LBD has a structure that resembles the agonist-bound, transcriptionally active LBD in many nuclear receptors, the NURR1 LBD contains no cavity that is capable of binding a ligand because of folding of its AF2 helix domain into the LBD. The large hydrophobic side chains of the AF2 domain appear to fill the LBD which suggests that the LBD inaccessible to binding by a ligand. In addition, NURR1 does not appear to contain a classical binding site for either coactivators or corepressors.

NURR1 is expressed predominantly in the dopaminergic neurons of the brain where it has a pivotal role in proper development of dopamine neurons (Zetterstrom et al., Science 276: 248-250 (1997). NURR1 is an immediate early response gene which appears to have a role in cell proliferation, differentiation, and apoptosis. In osteoblasts of bone, NURR1 expression is increased as an immediate early gene in response to parathyroid hormone (PTH) (Tetradis et al., Endocrinol. 142: 663-670 (2001). Recently, Lammi et al. (Molec. Endocrinol. Dol: 10.1210/me.2003-0247 (Feb. 26, 2004)) provide evidence that NURR1 might have a role in regulation of bone homeostasis. The authors show that NURR1 regulates osteoponin (OPN) expression in osteoblastic cell lines. The activation of the OPN promoter was mediated by the monomeric form of NURR1 which bound the NBRE nucleotide sequence in the OPN promoter, and was dependent on the amino-terminal transactivation function (AF-1). The authors further showed that NURR1 activated the OPN promoter in a synergistic fashion with the vitamin D receptor (VDR) and that NURR1 activation of OPN expression was repressed by the estrogen-related receptors (ERRs). As will be shown herein, NURR1 expression is elevated in normal prostate tissue from humans and rats and in cartilage tissue of rats in which osteoarthritis had been induced. Thus, NURR1 appears to have a role in regulation of bone homeostasis, development of osteoarthritis, and in prostate biology.

Published U.S. Patent Application No. U.S. 20020049151 to Murphy et al. discloses therapeutic approaches to diseases by suppression of the NURR subfamily of nuclear transcription factors. The NURR subfamily of transcription factors appear to have a central role in mediating multiple inflammatory signals. In particular, the nuclear receptors NURR1, NUR77, and NOR1 and have a role in modulating peripheral CRH and CRH-mediated signaling, which is an important component of inflammatory processes such as in human arthritis. The application discloses methods for treating inflammatory diseases such as inflammatory joint disease, ulcerative colitis, and thyroiditis, in particular arthritic diseases such as rheumatoid arthritis, psoriatic arthritis, and sarcoid arthritis by providing an antagonist that inhibits transcriptional activity of a NURR family nuclear receptor.

Published U.S. Patent Application No. 20030119026 to Lee and Vassilatis discloses mutations in NURR1 which provide molecular tools for the development of diagnostic, prophylactic, and therapeutic agents for Parkinson's Disease (PD). In specific embodiments, two point mutations were identified in exon 1 of the NURR1 gene in 9.3% cases of familial PD. The mutations reduced NURR1 gene expression by 87 to 95% and decreased tyrosine hydroxylase (a rate-limited dopamine synthesis enzyme) gene expression in vitro. The application also demonstrated that in vivo, NURR1 mRNA levels in lymphocytes from the PD patients with the mutation were reduced by 68 to 84%, and in over 50% of sporadic PD patients, the NURR1 mRNA levels in lymphocytes were significantly reduced. A homozygous polymorphism was identified in intron 6 of NURR1 that correlated with the presence of PD.

WO0100807 to Buervenich et al. discloses mutated NURR1 molecules that are useful as models for the study of psychotic disorders, such as schizophrenia or bipolar disorder, as well as for the identification of effective therapies and drugs for the treatment of the disorders.

U.S. Pat. No. 6,312,949 to Sakurada et al discloses methods and materials involved in the regulation of tyrosine hydroxylase expression as well as the treatment of catecholamine-related diseases. In particular, the invention provides cells that contain exogenous nucleic acid having a nucleic acid sequence that encodes NURR1 as well as methods and materials for inducing tyrosine hydroxylase expression, treating catecholamine-related deficiencies, and identifying tyrosine hydroxylase-related deficiencies.

Of interest are the following patents and applications: WO0187923 to Bresihan et al., WO0183715 to Lee et al., WO0100807 to Anvret et al., WO0066713 to Arenas et al., WO9826063 to Drouin et al., WO9404675 to Kroczek et al., WO 0170254 to Cairns et al., WO0077202 to Kremer et al., U.S. Pat. No. 6,284,539 to Bowen et al., and U.S. Pat. No. 5,874,534 to Vegeto et al.

In light of the role NURR1 appears to have in regulation of bone homeostasis, development of inflammatory diseases such as osteoarthritis, in prostate biology, and in various neurological disorders such as Parkinson's disease, and psychotic disorders such as bi-polar disorders, there is a need to provide methods for identifying compounds that modulate NURR1 activity.

### BRIEF SUMMARY OF THE INVENTION

The present invention provides the Macaca mulatta (rhesus monkey) NURR1 nuclear receptor (rhNURR1) and the nucleic acid encoding the rh NURR1. The present invention further provides methods for identifying analytes which modulate expression or activity of NURR1 for treating or inhibiting inflammatory diseases such as osteoarthritis, bone disorders, neurological and psychotic disorders, and prostate disorders.

Therefore, the present invention provides an isolated nucleic acid molecule comprising a nucleotide sequence encoding an rhNURR1 polypeptide or fragment thereof having the amino acid sequence shown in SEQ ID NO:2 or portion thereof. In a further aspect of the invention, the nucleic acid molecule encoding the rhNURR1 comprises the nucleotide sequence of SEQ ID NO:1. The present invention further provides an isolated nucleic acid encoding the LBD or the DBD of the rhNURR1 polypeptide. For example, the present invention provides a nucleic acid encoding the LBD, which comprises the nucleotides from about 987 to 1794 of SEQ ID NO:1, and a nucleic acid encoding the DBD, which comprises the nucleotides from about 1 to 986 of SEQ ID NO:1. The present invention further provides an isolated polypeptide comprising an amino acid sequence of SEQ ID NO:2.

The present invention further provides an antibody which binds a polypeptide comprising an amino acid sequence of SEQ ID NO:2. In further aspects, the antibody is a polyclonal antibody or a monoclonal antibody.

The present invention further still provides a vector comprising a nucleic acid encoding an rhNURR1 polypeptide or fragment thereof which has an amino acid sequence of SEQ ID NO:2. Preferably, the nucleic acid encoding the rhNURR1 comprises the sequence of SEQ ID NO:1. In particular aspects of the vector, the vector comprises a nucleic acid encoding the LBD or the DBD of the rhNURR1. In further aspects of the above vector, the nucleic acid encodes a fusion or chimeric protein, which comprises the rhNURR1 polypeptide, the LBD of the rhNURR1, or the DBD of the NURR1 fused to a heterologous protein. In particular aspects of the vector, the fusion protein comprises the rhNURR1 LBD fused to the GAL4 yeast transcription activation factor DBD or glucocorticoid receptor (GR) DBD; or, the H1 alpha helix of the NURR1 LBD fused to the GAL4 or GR LBD and the remainder of the NURR1 LBD fused to the herpesvirus VP16 activation domain. In a further still aspect of the above vector, the present invention further provides a vector wherein the nucleic acid encoding the rhNURR1 polypeptide or fragment thereof, or fusion protein, is operably linked to a heterologous promoter. The heterologous promoter can be a constitutive promoter or an inducible promoter.

The present invention further still provides a cell comprising a nucleic acid encoding an rhNURR1 polypeptide or fragment thereof having an amino acid sequence of SEQ ID NO:2 wherein the nucleic acid is operably linked to a heterologous constitutive or inducible promoter which enables expression of the rhNURR1 or fragment thereof in the cell. Preferably, the nucleic acid comprises the sequence of SEQ ID NO:1 or a subsequence thereof. For example, the nucleic acid can comprise nucleotides from about 987 to 1794 of SEQ ID NO:1, which encode the rhNURR1 LBD, or nucleotides from about 1 to 986 of SEQ ID NO:1, which encode the rhNURR1 DBD.

In a further aspect of the above cell, the nucleic acid encodes a fusion or chimeric protein comprising the rhNURR1 polypeptide, the NURR1 LBD or the NURR1 DBD fused to a heterologous protein. In particular aspects of the above cell, the fusion protein comprises the rhNURR1 LBD fused to the GAL4 yeast transcription activation factor DBD or GR DBD; or, the H1 alpha helix of the LBD fused to the GAL4 or GR LBD and the remainder of the LBD fused to the herpesvirus VP 16 activation domain. In a further still aspect of the above cell, the present invention provides a vector wherein the nucleic acid encoding the rhNURR1 polypeptide or fragment thereof, or fusion protein, is operably linked to a heterologous constitutive or inducible promoter.

In a further aspect of the above cell, the cell further includes a second nucleic acid encoding a reporter gene, which encodes an assayable product, wherein the nucleic acid encoding the reporter gene is operably linked to a promoter responsive to the rhNURR1. In further aspects of the above cell, the promoter responsive to the rhNURR1 includes at least one NGFI-B response element (NBRE). Examples of promoters responsive to the rhNURR1 include the gene encoding osteoponin (OPN), tyrosine hydroxylase (TH), or human dopamine transporter (DAT).

In a further still aspect of the above cell, the cell expresses an endogenous retinoid X receptor (RXR) or includes a third nucleic acid which encodes the RXR for ectopic expression of the RXR.

The present invention further still provides a method for producing an rhNURR1 polypeptide or fragment thereof comprising (a) providing a nucleic acid encoding the rhNURR1 polypeptide operably linked to a heterologous promoter; (b) introducing the nucleic acid into a cell to produce a recombinant cell; and (c) culturing the recombinant cell under conditions suitable for expression of the nucleic acid encoding the rhNURR1 to produce the rhNURR1 polypeptide or fragment thereof. Preferably, the nucleic acid comprises the nucleotide sequence of SEQ ID NO:1 or subsequence thereof. For example, a nucleic acid fragment is provided which encodes the rhNURR1 LBD (nucleotides from about 987 to 1794) or the DBD (nucleotides from about 1 to 986).

In further aspects of the above method, the nucleic acid encodes a fusion or chimeric protein comprising the NURR1 polypeptide, the LBD or the DBD fused to a heterologous protein. In particular aspects of the above method, the fusion protein comprises the rhNURR1 LBD fused to the GAL4 yeast transcription activation factor DBD or GR DBD; or, the H1 helix of the LBD fused to the GAL4 or GR LBD and the remainder of the LBD fused to the herpesvirus VP16 activation domain. In further still aspects of the above methods, the present invention further provides a vector wherein the nucleic acid encoding the rhNURR1 polypeptide or fragment thereof, or fusion protein, is operably linked to a heterologous promoter. In further still aspects of the above method, the cell is a selected from the group consisting of mammalian cells, prokaryote cells, insect cells, fungal cells, and plant cells. In particular aspects of the above method, the recombinant cell is either transiently transfected with the nucleic acid or is stably transfected cell with the nucleic acid.

The present invention further still provides a method for identifying an analyte that modulates activity of an rhNURR1, which comprises (a) providing a recombinant cell that expresses the rhNURR1; (b) incubating the recombinant cell in a medium which includes the analyte; and (c) measuring activity of the rhNURR1, wherein a change in the activity of the rhNURR1 in the presence of the analyte indicates the analyte modulates the activity of the rhNURR1. Preferably, the rhNURR1 is encoded by a nucleic acid which comprises the nucleotide sequence of SEQ ID NO:1.

In further aspects of the above method, the recombinant cell is a selected from the group consisting of mammalian cells, prokaryote cells, insect cells, fungal cells, and plant cells. In particular aspects of the above method, the recombinant cell is a mammalian cell which is either transiently or stably transfected with a nucleic acid encoding the rhNURR1. Preferably, the rhNURR1 is encoded by a nucleic acid comprising the nucleotide sequence of SEQ ID NO:1. In a further aspect of the above method, the activity of the rhNURR1 is determined by measuring expression of a reporter gene operably linked to a promoter which is responsive to rhNURR1. In particular aspects of the above method, the rhNURR1 responsive promoter includes at least one NGFI-B response element (NBRE). Examples of rhNURR1 responsive promoters include the gene encoding OPN, TH, or DAT. In further aspects of the above method, the reporter gene encodes luciferase or SEAP.

The present invention further provides a method for identifying an analyte that modulates activity of an rhNURR1, which comprises (a) providing a recombinant cell which expresses the rhNURR1 and an assayable reporter gene product wherein the reporter gene encoding the product is operably linked to a promoter responsive to the rhNURR1; (b) incubating the recombinant cell in a medium which includes an analyte; and (c) measuring the expression of the reporter gene product wherein an increase or decrease of expression of the reporter gene compared to expression in a recombinant cell in the absence of the analyte identifies the analyte that modulates activity of the rhNURR1. Preferably, the rhNURR1 is encoded by a nucleic acid which comprises the nucleotide sequence of SEQ ID NO:1.

In particular aspects of the above method, the recombinant cell further expresses a retinoid X receptor (RXR). The expression of the RXR can be ectopic or endogenous. In further still aspects, the activity of the rhNURR1 is determined by measuring expression of a reporter gene encoding an assayable product which is operably linked to a promoter that is responsive to rhNURR1, e.g., a promoter which includes at least one NBRE binding site. In further aspects of the above method, the reporter gene encodes luciferase or SEAP. In particular aspects of the above method, the recombinant cell is a mammalian cell either transiently or stably transfected with nucleic acids encoding the rhNURR1 and reporter gene.

The present invention further still provides a method for identifying an analyte that modulates heterodimerization between an rhNURR1 and an RXR, which comprises (a) providing a recombinant cell which expresses the rhNURR1 and an assayable reporter gene product wherein the reporter gene encoding the product is operably linked to a promoter responsive to the rhNURR1/RXR heterodimer; (b) incubating the recombinant cell in a medium which includes an analyte; and (c) measuring the expression of the reporter gene product wherein an increase or decrease of expression of the reporter gene compared to expression in a recombinant cell in the absence of the analyte identifies the analyte that modulates the heterodimerization between the rhNURR1 and the RXR. Preferably, the rhNURR1 is encoded by a nucleic acid which comprises the nucleotide sequence of SEQ ID NO:1.

In particular aspects of the above method, the RXR expression is endogenous to the cell and in other aspects, the expression is ectopic. In further aspects of the above method, the reporter gene encodes luciferase or SEAP. In particular aspects of the above method, the recombinant cell is a mammalian cell either transiently or stably transfected with nucleic acids encoding the rhNURR1 and reporter gene.

In any one of the above methods, the rhNURR1 can be provided as a fusion or chimeric protein comprising the LBD of the rhNURR1 (encoded by the nucleotides from about 987 to 1794 of SEQ ID NO:1) fused to a heterologous protein. In particular aspects of the above methods, the fusion protein comprises the rhNURR1 LBD fused to the GAL4 yeast transcription activation factor DBD or GR DBD.

The present invention further still provides a method for identifying an analyte that stabilizes an rhNURR1 LBD, which comprises (a) providing a cell that does not stabilize the LBD of the rhNURR1; (b) transfecting the cell with a first gene expression cassette encoding a first fusion protein comprising the H1 alpha helix domain of the LBD of the rhNURR1 fused to a heterologous DBD, a second gene expression cassette encoding a second fusion protein comprising the remainder of the LBD of the NURR1 fused to a heterologous transcription activation domain, and a third gene expression cassette encoding an assayable reporter gene product wherein the reporter gene is operably linked to a promoter which binds the heterologous DBD and is activated by the heterologous activation domain to produce a recombinant cell; (b) incubating the recombinant cell in a medium which includes an analyte; and (c) measuring the expression of the reporter gene product wherein expression of the reporter gene product indicates the analyte stabilizes the LBD of the rhNURR1.

The present invention further still provides a method for identifying an analyte that destabilizes the rhNURR1 LBD, which comprises (a) providing a cell that stabilizes the LBD of the rhNURR1; (b) transfecting the cell with a first gene expression cassette encoding a first fusion protein comprising the H1 domain of the LBD of the rhNURR1 fused to a heterologous DBD, a second gene expression cassette encoding a second fusion protein comprising the remainder of the LBD of the NURR1 fused to a heterologous transcription activation domain, and a third gene expression cassette encoding an assayable reporter gene product wherein the reporter gene is operably linked to a promoter which binds the heterologous DBD and is activated by the heterologous activation domain to produce a recombinant cell; (b) incubating the recombinant cell in a medium which includes an analyte; and (c) measuring the expression of the reporter gene product wherein no expression of the reporter gene product indicates the analyte destabilizes the LBD of the rhNURR1. Preferably, the rhNURR1 H1 domain is encoded by a nucleic acid which comprises the nucleotide sequence from about 1050 to 1194 of SEQ ID NO:1 and the remainder of the LBD is encoded by a nucleic acid which comprises the nucleotide sequence from about 1195 to about 1794.

In particular aspects of the above methods, the heterologous DBD is the GAL4 yeast transcription factor DBD or the GR DBD. In further aspects, the heterologous transcription activation domain is the herpes simplex VP16 transcription activation domain. In further still aspects, the reporter gene encodes luciferase or SEAP. In particular aspects, the recombinant cell is a mammalian cell either transiently or stably transfected with the gene expression cassettes. In a further aspect of the former method, the cell is a human embryonic kidney 293 cell and in the latter method, the cell is a human chorion carcinoma JEG-3 cell.

The present invention further provides a method for identifying an analyte useful for inducing NURR1 expression in a prostate cancer cell, which comprises (a) providing a prostate cancer cell; (b) incubating the cell in a medium which includes an analyte; and, (c) measuring expression of NURR1 in the cell, wherein an increase in expression of NURR1 in the cell in the presence of the analyte compared to expression of the NURR1 in the cell in the absence of the analyte indicates that the analyte is useful for treating the prostate cancer in the mammal.

The present invention further provides a method for identifying an analyte useful for treating prostate cancer in a mammal, which comprises (a) providing a prostate cancer cell; (b) incubating the cell in a medium which includes an analyte; and, (c) measuring expression of NURR1 in the cell, wherein an increase in expression of NURR1 in the cell in the presence of the analyte compared to expression of the NURR1 in the cell in the absence of the analyte indicates that the analyte is useful for treating the prostate cancer in the mammal.

The present invention further provides a method for treating prostate cancer in a mammal, which comprises (a) providing a prostate cancer cell; (b) incubating the cell in a medium which includes an analyte; and (c) measuring expression of NURR1 in the cell to identify an analyte that induces expression of NURR1 in the prostate cancer cell; and (d) administering the analyte identified in step (c) to the mammal to treat the prostate cancer.

In a further aspect of the above three methods, the prostate cancer cell is selected from the group consisting of 22Rv1 cells and LNCap cells. In a further still aspect, the expression of the NURR1 is determined by measuring the amount of RNA encoding the NURR1 in the cell. In a further still aspect, the amount of RNA is measured by reverse-transcription polymerase chain reaction. In a further still aspect, the expression of the NURR1 is determined by measuring the amount of NURR1 polypeptide in the cell. In a further still aspect, the amount of NURR1 polypeptide is determined by using an antibody specific for the NURR1.

The present invention further provides a method for treating prostate cancer in a mammal, which comprises (a) obtaining prostate cancer cells from the mammal; (b) providing a multiplicity of cultures of the cells wherein each of the cultures of the cells is incubated in a medium that includes an analyte to be tested for ability to induce NURR1 expression in the cells; (c) measuring expression of NURR1 in each of the cultures of cells to identify an analyte that induces the NURR1 expression in the cells; and (d) administering the analyte that induces the NURR1 expression in the cells to the mammal to treat the prostate cancer in the mammal.

In a further aspect of the above method, the expression of the NURR1 is determined by measuring the amount of RNA encoding the NURR1 in the cell. In a further still aspect, the amount of RNA is measured by reverse-transcription polymerase chain reaction. In a further still aspect, the expression of the NURR1 is determined by measuring the amount of NURR1 polypeptide in the cell. In a further still aspect, the amount of NURR1 polypeptide is determined by using an antibody specific for the NURR1.

The present invention further provides a method for identifying an analyte useful for treating osteoarthritis in a mammal, which comprises: (a) inducing osteoarthritis in a rat; (b) providing the analyte to the rat; and, (c) measuring expression of NURR1 in the cells of the knees of the osteoarthritic rat, wherein a decrease in expression of the NURR1 in the cells of the rat knees treated with the analyte indicates that the analyte is useful for treating the osteoarthritis in the mammal.

The present invention further provides a method for identifying an analyte that suppresses NURR1 expression in the cells of a joint from a mammal with osteoarthritis, which comprises: (a) inducing osteoarthritis in a rat; (b) providing the analyte to the rat; and, (c) measuring expression of NURR1 in the chondrocytes and synovial cells of the knees of the osteoarthritic rat, wherein a decrease in expression of the NURR1 in the chondrocytes and synovial cells of the knees mouse treated with the analyte indicates that the analyte suppresses NURR1 expression in the cells.

The present invention further provides a method for treating osteoarthritis in a mammal, which comprises: (a) inducing osteoarthritis in a multiplicity of rats; (b) providing to each of the mice an analyte to be tested for ability to suppress NURR1 expression in the cells of the joints of the rats; and, (c) measuring expression of NURR1 in the cells of the knees of the osteoarthritic rat to identify an analyte which suppresses NURR1 expression in the cells; and, (d) administering the analyte identified in (c) to the mammal to treat the osteoarthritis.

In a further aspect of the above two methods, the osteoarthritis is induced in the rat by a chemical treatment, resistance training, or a surgical treatment. In a further still aspect, the NURR1 expression is determined in the cells obtained from an anterior cruciate ligament transection or ACL transection and medial meniscetomy of the knees from the osteoarthritic rat. In a further aspect of the method, the expression of the NURR1 is determined by measuring the amount of RNA encoding the NURR1 in the cells. In a further still aspect, the amount of RNA is measured by reverse-transcription polymerase chain reaction. In a further aspect of the method, the expression of the NURR1 is determined by measuring the amount of NURR1 polypeptide in the cells. In a further still aspect, the amount of NURR1 polypeptide is determined by using an antibody specific for the NURR1. In a further aspect, the cells are selected from the group consisting of chondrocytes and synovial cells.

As used throughout the specification and appended claims, the following definitions and abbreviations apply.

The term “rhNURR1” means that the rhNURR1 is of Macaca mulatta (rhesus monkey) origin, either isolated from rhesus monkey tissue, produced from a nucleic acid obtained from the rhesus monkey by recombinant means, produced from a nucleic acid synthesized in vitro but which encodes the rhNURR1, or synthesized in vitro. The term further includes biologically active fragments or portions of the rhNURR1, including fusion or chimeric proteins.

The term “NURR1” means that the NURR1 is not of rhesus monkey origin. The NURR1 can be from another organism, for example, a mammal such as rat and mouse, and humans. The NURR1 can either be isolated from tissue of the organism, produced from a nucleic acid obtained from the organism by recombinant means, produced from a nucleic acid synthesized in vitro but which encodes the NURR1, or synthesized in vitro. The term further includes biologically active fragments or portions of the NURR1, including fusion or chimeric proteins.

The term “NURR1 binding partner” means a binding partner which can bind rhNURR1 or NURR1 regardless of whether it originates from the rhesus monkey, another organism, expressed from a nucleic acid synthesized in vitro, or synthesized in vitro. Examples of NURR1 binding partners include RXR, ligands, co-activators, and nucleic acids which comprise an NBRE binding site. The term further includes biologically active fragments or portions of the NURR1 binding partner.

The term “homologous NURR1 binding partner” means a NURR1 binding partner of rhesus monkey origin. In other words, the NURR1 binding partner is isolated from the rhesus monkey or obtained from recombinant cells expressing the binding partner from DNA encoding the rhesus monkey binding partner.

The term “heterologous NURR1 binding partner” means a NURR1 binding partner not of rhesus monkey origin. For example, the NURR1 binding partner is isolated from a human or obtained from recombinant cells expressing the binding partner from DNA encoding the human binding partner.

The term “promoter” refers to a recognition site on a DNA strand to which RNA polymerase binds. The promoter forms an initiation complex with RNA polymerase to initiate and drive transcriptional activity of a nucleic acid sequence located downstream from the promoter. The promoter can be modified by including activating sequences termed “enhancers” or inhibiting sequences termed “silencers” within the promoter. The term further includes both promoters which are inducible and promoters which are constitutive.

The term “cassette” refers to a nucleotide or gene sequence that is to be expressed from a vector, for example, the nucleotide or gene sequence encoding the rhNURR1 or RXR. In general, a cassette comprises a gene sequence inserted into a vector which in some embodiments provides regulatory sequences for expressing the nucleotide or gene sequence. In other embodiments, the nucleotide or gene sequence provides the regulatory sequences for its expression. In further embodiments, the vector provides some regulatory sequences and the nucleotide or gene sequence provides other regulatory sequences. For example, the vector can provide a promoter for transcribing the nucleotide or gene sequence and the nucleotide or gene sequence provides a transcription termination sequence. The regulatory sequences which can be provided by the vector include, but are not limited to, enhancers, transcription termination sequences, splice acceptor and donor sequences, introns, ribosome binding sequences, and poly(A) addition sequences.

The term “vector” refers to a means by which DNA fragments can be introduced into a host organism or host tissue. There are various types of vectors including plasmid, virus (including adenovirus, herpesvirus, and the like), bacteriophage, and cosmid.

The term “substantially free from other nucleic acids” means at least 90%, preferably 95%, more preferably 99%, and even more preferably 99.9%, free of other nucleic acids. As used interchangeably, the terms “substantially free from other nucleic acids,” “substantially purified,” “isolated nucleic acid” or “purified nucleic acid” also refer to DNA molecules which comprise a coding region for an rhNURR1 polypeptide that has been purified away from other cellular components. Thus, an rhNURR1 DNA preparation that is substantially free from other nucleic acids will contain, as a percent of its total nucleic acid, no more than 10%, preferably no more than 5%, more preferably no more than 1%, and even more preferably no more than 0.1%, of non-rhNURR1 nucleic acids. Whether a given rhNURR1 DNA preparation is substantially free from other nucleic acids can be determined by such conventional techniques of assessing nucleic acid purity such as agarose gel electrophoresis combined with an appropriate staining method such as ethidium bromide staining, or by sequencing.

The term “substantially free from other polypeptides” or “substantially purified” means at least 90%, preferably 95%, more preferably 99%, and even more preferably 99.9%, free of other proteins. Thus, an rhNURR1 protein preparation that is substantially free from other proteins will contain, as a percent of its total protein, no more than 10%, preferably no more than 5%, more preferably no more than 1%, and even more preferably no more than 0.1%, of non-rhNURR1s. Whether a given rhNURR1 preparation is substantially free from other proteins can be determined by such conventional techniques of assessing protein purity as, e.g., sodium dodecyl sulfate polyacrylamide gel electrophoresis (SDS-PAGE) combined with appropriate detection methods, for example, silver staining or immunoblotting.

As used interchangeably, the terms “substantially free from other proteins” or “substantially purified,” or “isolated rhNURR1” or “purified rhNURR1” also refer to rhNURR1 which has been isolated from a natural source. Use of the term “isolated” or “purified” indicates that rhNURR1 has been removed from its normal cellular environment. Thus, an isolated rhNURR1 may be in a cell-free solution or placed in a different cellular environment from that in which it occurs naturally. The term isolated does not imply that an isolated rhNURR1 is the only protein present, but instead means that an rhNURR1 is substantially free of other proteins and non-amino acid material (for example, nucleic acids, lipids, carbohydrates) naturally associated with the rhNURR1 in vivo. Thus, a rhNURR1 that is recombinantly expressed in a prokaryotic or eukaryotic cell and substantially purified from this host cell and which does not naturally (that is, without intervention) express this rhNURR1 is an “isolated rhNURR1” under any of circumstances referred to herein. As noted above, a rhNURR1 preparation that is an isolated or purified rhNURR1 will be substantially free from other proteins and will contain, as a percent of its total protein, no more than 10%, preferably no more than 5%, more preferably no more than 1%, and even more preferably no more than 0.1%, of non-rhNURR1.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid).

The term “agonist” refers to an agent that upregulates (for example, turns on, enhances, potentiates, or supplements) or mimics rhNURR1 bioactivity. In one aspect, an agonist can be an analyte that turns on or upregulates activity of rhNURR1 from an inactive state to an active state. For example, an agonist can be an analyte that when bound to the rhNURR1 enables or enhances the binding of the rhNURR1 to its binding partner, e.g., RXR or a NURR1 responsive promoter. In another aspect, an agonist can be an analyte that mimics a bioactivity of rhNURR1, such as transduction of a signal from a NURR1 binding partner when bound to the binding partner. In a further aspect, an agonist can be an analyte that upregulates expression of rhNURR1. In a further still aspect, an agonist can be an analyte that modulates the expression or activity of a protein, which is located downstream, for example, of a binding partner, thereby mimicking or enhancing the effect of binding of rhNURR1 to the binding partner.

The term “antagonist” refers to an agent that inhibits, decreases, or suppresses a bioactivity of rhNURR1. In one aspect, an antagonist can be an analyte that decreases signaling from rhNURR1, for example, an analyte that is capable of binding to rhNURR1 or a NURR1 binding partner. A preferred antagonist inhibits or suppresses rhNURR1 bioactivity either by interacting with or binding to the rhNURR1 or by interacting with or binding to a NURR1 binding partner in a manner that suppresses the interacting or binding of the rhNURR1 to the binding partner. In another aspect, an antagonist can be an analyte that downregulates expression of the rhNURR1. In a further aspect, an antagonist can also be an analyte that modulates the expression or activity of a protein which is located downstream of the rhNURR1, thereby antagonizing the effect of binding of rhNURR1 to its binding partner.

A “disorder” is any condition that would benefit from treatment with analytes identified by the methods described herein. This includes chronic and acute disorders or diseases including those pathological conditions which predispose the mammal to the disorder in question.

The term “mammalian” refers to any mammal, including a human being.

The term “treatment” refers to both therapeutic treatment and prophylactic or preventative measures. Those in need of treatment include those already with the disorder as well as those prone to have the disorder or those in which the disorder is to be prevented.

The term “SARM” refers to selective androgen receptor modulator.

### BRIEF DESCRIPTION OF THE DRAWINGS

FIG. 1 shows the nucleotide sequence for cDNA encoding rhNURR1 (SEQ ID NO:1).

FIG. 2 shows the amino acid sequence for rhNURR1 (SEQ ID NO:2). The region encompassing the LBD is underlined.

FIGS. 3A, 3B, and 3C show a nucleotide sequence alignment comparison between the rhNURR1 (SEQ ID NO:1) and the human NURR1 (SEQ ID NO:3).

FIG. 4A shows the relative expression profile of human NURR1 in a variety of human tissues.

FIG. 4B shows the relative expression profile of rat NURR1 in a variety of rat tissues.

FIG. 5 shows that NURR1 expression is elevated in osteoarthritis-induced articular cartilage of rats. Osteoarthritis was induced in the right knee of the rats by surgically induced joint instability. Osteoarthritis was assessed in the rat knees subjected to either anterior cruciate ligament (ACL) transection or ACL transection and medial meniscetomy (MM), harvested one, two, or four weeks postsurgery.

FIG. 6 shows the relative transactivation of a reporter gene operably linked to the MMTV promoter by a chimeric receptor consisting of the glucocorticoid DBD and the NURR1 LBD in response to several compounds.

FIG. 7 shows the relative transactivation of GR/NURR1 chimera receptor in response to various compounds. D is Bicalutamide, E is Lovastatin, and F is ZOCOR. The remainder are various SARM compounds.

FIG. 8 shows the relative transactivation of GR/NURR1 chimera receptor in response to several compounds. D is Bicalutamide, E is Lovastatin, F is ZOCOR. The remainder are various SARM compounds.

FIG. 9 shows the relative NURR1 expression in prostate cancer cells 22Rv1 and LNCap compared to expression in breast cancer cells MDA. The red bar is the DMSO vehicle, the yellow bar is dexamethasone, the light green bar is dexamethasone, the blue bar is bicalutamide, the dark green bar is SARM compound A, the purple bar is SARM compound J, and the violet bar is SARM compound K.

### DETAILED DESCRIPTION OF THE INVENTION

The present invention provides nucleic acid molecules which encode the Macaca mulatta (rhesus monkey) NURR1 (rhNURR1) and the rhNURR1 polypeptide encoded by the nucleic acid. The present invention further provides methods for using the nucleic acid molecules and the rhNURR1 produced therefrom in assays for identifying analytes (molecules, compounds, drugs, or compositions) which modulate (interfere with, alter, enhance, stimulate, repress, inhibit, or suppress) the activity of rhNURR1 as an agonist, antagonist, or tissue selective ligand. In particular, the present invention provides assays for identifying analytes which modulate NURR1 activation of transcription from a NURR1 responsive promoter as a monomer, homodimer, or a heterodimer with RXR or other binding partner.

NURR1 responsive promoters include promoters that comprise one or more NGFI-B response elements or binding sites (NBRE; 5′-AAAGGTCA-3′; SEQ ID NO:6) which NURR1 can bind as a monomer. For example, the promoter for the osteopontin gene is a NURR1 responsive promoter that comprises NBRE binding sites. Other promoters responsive to NURR1 include the promoter for the tyrosine hydroxylase (TH) gene, which comprises an NBRE binding site and several NBRE-like binding sites, (Kim et al., J. Neurochem. 85: 622-634 (2003) and the promoter for the pro-opiomelanocortin gene, which comprises the palindromic nucleotide sequence 5′-TGACCTTT-N₃-AAAGGTCA-3′ (SEQ ID NO:4) that NURR1 binds as a homodimer (Maira et al., Molec. Cell. Biol. 19: 7549-7557 (1999). NURR1 responsive promoters further include promoters which provide for NBRE-independent NURR1 activation, for example, the promoter for the human dopamine transporter (DAT) gene, (Sacchetti et al., J. Neurochem. 76: 1565-1572 (2001), or promoters that comprise binding sites which are recognized by the NURR1—RXR heterodimer such as the nucleotide sequence 5′-AGGTCA-N-3-AAAGGTCA (SEQ ID NO:5) which is recognized by the NURR1—RXR heterodimer in the presence of 9-cis retinoic acid (Perlmann and Jansson, Genes Dev. 9: 769-782 (1995).

The methods further include methods or assays for identifying analytes which modulate the molecular or functional interaction between the rhNURR1 and one or more homologous or heterologous NURR1 binding partners (for example, a protein binding partner such as retinoid X receptor (RXR) or a nucleic acid binding partner such as a nucleic acid comprising at least one NBRE binding site) and analytes which affect the ability of the rhNURR1 or rhNURR1/protein binding partner complex to activate transcription at a promoter responsive to the NURR1 or NURR1/protein binding partner complex. Non-limiting examples of methods for identifying such analytes include (i) cell-based assays for identifying analytes which inhibit or suppress the interaction or binding between rhNURR1 and one or more homologous or heterologous NURR1 binding partners such as RXR protein expressed in mammalian cells or transcriptional activation of a promoter responsive to NURR1 activation; and (ii) cell-free binding assays for identifying analytes which inhibit or suppress (a) binding between rhNURR1 and one or more homologous or heterologous protein binding partners such as RXR protein, (b) binding to a nucleic acid comprising a NURR1 binding site such as the NBRE binding site, or (c) transcriptional activation of a promoter responsive to NURR1 activation. Thus, the methods described herein are useful tools for identifying analytes which modulate molecular and/or functional interactions between NURR1 and one or more homologous or heterologous binding partners or activation of a responsive promoter.

NURR1 is expressed in the dopaminergic neurons of the brain, where it has a pivotal role in the proper development of dopamine neurons (Zetterstrom et al., Science 276: 248-250 (1997)). NURR1 regulates transcription of the gene encoding the cocaine-sensitive dopamine transporter (DAT), a plasma membrane transport protein that regulates extracellular dopamine concentrations. Deletion of NURR1 resulted in failure to generate midbrain dopaminergic neurons. NURR1 also modulates neuroendocrine regulation of the hypothalamic pituitary adrenal axis.

Tetradis et al. (Endocrinol. 142: 663-670 (2001)) have shown that in osteoblasts of bone, NURR1 expression is increased as an immediate early gene in response to parathyroid hormone (PTH) and Lammi et al. (Molec. Endocrinol. Dol: 10.1210/me.2003-0247 (Feb. 26, 2004)) provide evidence that suggests NURR1 has a role in regulation of bone homeostasis.

Lee and Vassilatis (Published U.S. Patent Application No. 20030119026) suggest that NURR1 has a role in Parkinson's Disease (PD) and Buervenich et al. (WO0100807) suggest that NURR1 has a role in psychotic disorders, such as schizophrenia or bipolar disorder, as well as for the identification of effective therapies and drugs for the treatment of the disorders.

Murphy et al., (Arthritis Rheum 44: 782-793 (2001)) has shown that NURR1 is involved in regulation of corticotrophin-releasing hormone expression and activity in inflammatory arthritis and Murphy et al., (J. Immunol. 168:2979-2987 (2002)) provide data which suggests that because NURR1 induction appears to be common to at least two distinct signaling pathways, NURR1 has a common role in mediating multiple inflammatory signals.

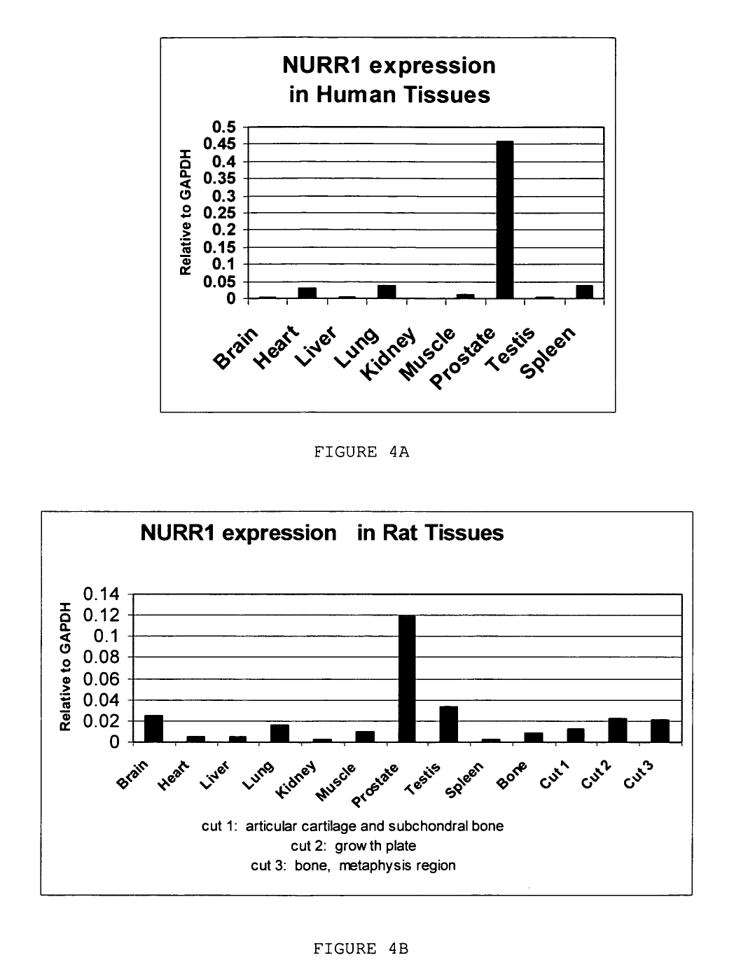

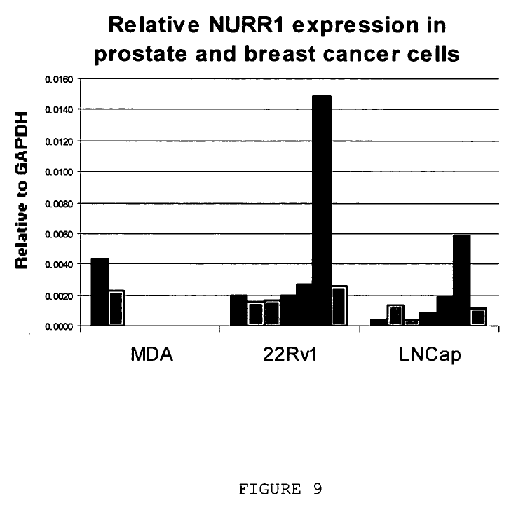

By screening a panel of tissues, we discovered that NURR1 is expressed in prostate tissue from normal males. As shown in FIG. 4A, NURR1 is highly expressed in prostate tissue from normal men while its expression in tissue from the brain, heart, liver, lung, muscle, testis, and spleen is detectable but significantly less than the expression in prostate tissue. FIG. 4B shows that NURR1 is highly expressed in prostate tissue from normal rats with detectable to moderate expression in testes, brain, lung, and bone, particularly, in the articular cartilage and subchondral bone, the growth plate bone, and the metaphysis region of bone. It was then found that a SARM that inhibited proliferation of prostate cancer cells induced NURR1 expression in those cells (FIG. 9 shows induction of NURR1 in prostate cancer cell lines 22Rv1 and LNCap treated with a SARM). The high expression of NURR1 in normal prostate tissue suggests that NURR1 has a role in prostate biology, particularly when viewed in light of data showing that inducing expression of NURR1 in prostate cancer cells inhibited proliferation of the cells.

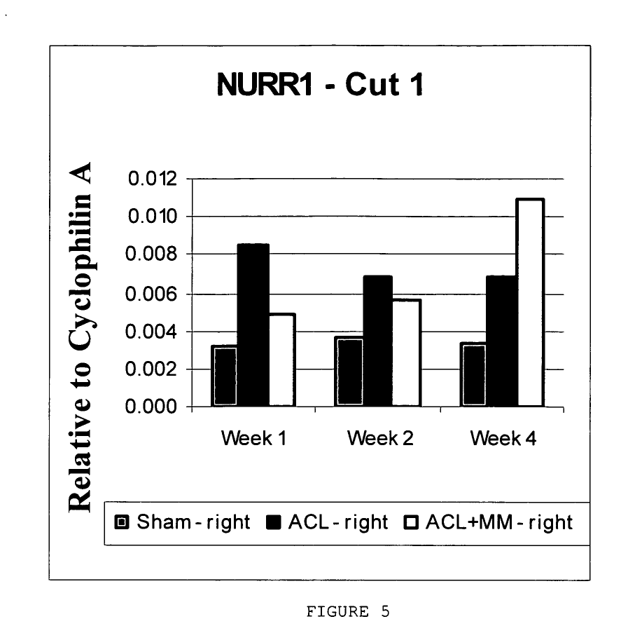

We also discovered that NURR1 appears to have a role in osteoarthritis. As shown in FIG. 5, NURR1 expression is induced at an early stage in osteoarthritic chondrocytes taken from rats in which osteoarthritis had been induced. The data suggests that modulating NURR1 expression or activity might provide an efficacious treatment for osteoarthritis.

In light of the above, analytes identified by the assays disclosed herein should be useful for treating a wide variety of diseases and disorders. For example, analytes identified by the assays disclosed herein will be useful for treating or controlling inflammatory diseases, in particular, inflammatory diseases such as inflammatory joint disease, ulcerative colitis, and thyroiditis, and arthritic diseases such as rheumatoid arthritis, osteoarthritis, psoriatic arthritis, and sarcoid arthritis. The assays disclosed here in will also be useful for identifying analytes for treating or controlling neurological disorders such as Parkinson's disease, bipolar disorder, and schizophrenia; analytes for treating various bone disorders such as osteoporosis; and, analytes for treating prostate diseases such as prostate hypertrophy or prostate cancer. The nucleic acid encoding the rhNURR1 is further useful for providing recombinant rhNURR1 (full-length or fragments comprising domains thereof such as the LBD or the DBD) for crystallographic structural studies on rhNURR1 or its domains and their interaction with various binding partners and analytes.

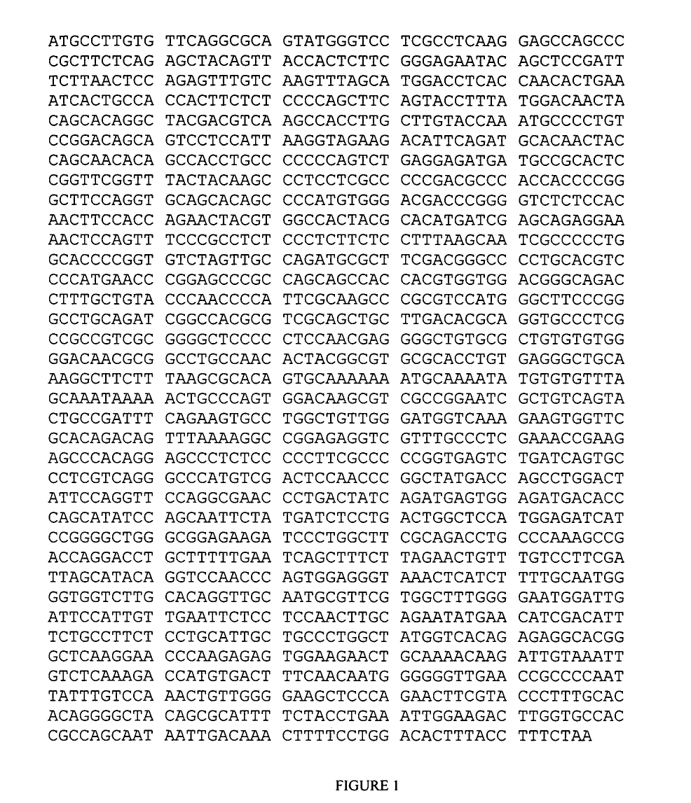

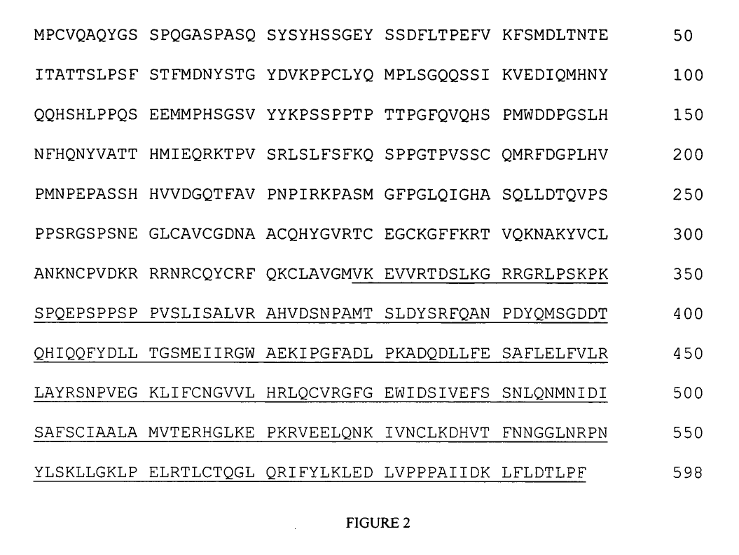

Therefore, in a first aspect, the present invention provides nucleic acid molecules which encode the rhNURR1. The isolated nucleic acid molecules include both deoxyribonucleic acid (DNA) and ribonucleic acid (RNA) molecules encoding the rhNURR1. The isolated nucleic acid molecules further include genomic DNA and complementary DNA (cDNA) encoding the rhNURR1, either of which can be single- or double-stranded, as well as synthetic DNA, such as a synthesized, single stranded polynucleotide. When single-stranded, the DNA molecule can comprise either the coding (sense) strand or the non-coding (antisense) strand. The nucleic acids encoding the rhNURR1 have a nucleotide sequence substantially similar to the nucleotide sequence set forth in SEQ ID NO:1 (FIG. 1). For most cloning purposes, DNA is the preferred nucleic acid. In the case of nucleic acid molecules isolated from genomic DNA, the nucleic acid sequences encoding the rhNURR1 comprising the amino acid sequence in SEQ ID NO:2 can be interrupted by one or more introns. Therefore, in a further aspect of the present invention, rhNURR1 polypeptides are provided which have an amino acid sequence which is substantially similar to the amino acid sequence set forth SEQ ID NO:2 (FIG. 2) and nucleic acids which encode the rhNURR1 polypeptides for use in the analyte screening assays disclosed herein.

As used herein, the term “substantially similar” with respect to SEQ ID NO:2 means that the rhNURR1 contains mutations such as amino acid substitution or deletion mutations which do not abrogate the ability of the rhNURR1 to bind at least one of its binding partners or activate transcription from a NURR1 responsive promoter. The mutations include naturally occurring allelic variants and variants produced by recombinant DNA methods. As used herein, the term “substantially similar” with respect to SEQ ID NO:1 means that the rhNURR1 encoded by the nucleic acid contains mutations such as nucleotide substitution or deletion mutations which do not abrogate the ability of the rhNURR1 to bind at least one of its binding partners or to activate transcription from a NURR1 responsive promoter. The mutations include naturally occurring allelic variants and variants produced by recombinant DNA methods. In general, any of the foregoing mutations which do not abrogate the ability of rhNURR1 to bind at least one of its homologous or heterologous binding partners or activate transcription from a NURR1 responsive promoter are conservative mutations.

The present invention further includes a nucleic acid which encodes a biologically active fragment or mutant of the rhNURR1. In general, the nucleic acid will encode either a polypeptide or polypeptide fragment, which at least substantially mimics the properties or activity of the rhNURR1 or a particular domain of the rhNURR1, for example, a nucleic acid encoding the ligand binding domain (LBD) from about nucleotide 1069 to about nucleotide 1794, the DNA binding domain (DBD) from about nucleotide 783 to about nucleotide 1008, the amino-terminal transactivation function 1 (AF1) from about nucleotide 1 to about nucleotide 252, the carboxy-terminal activation domain 2 (AF2) from about nucleotide 749 to about nucleotide 1794, the H1 alpha helix domain from about nucleotide 1050 to about nucleotide 1194, and the H3 to H12 alpha helix domains from about nucleotide 1197 to about nucleotide 1794. The above nucleic acid can comprise one or more nucleotide substitutions, deletions, additions, amino-terminal truncations, and carboxy-terminal truncations which do not substantially abrogate the properties or activities of the rhNURR1 or particular domain produced therefrom. Thus, the mutations of the present invention encode mRNA molecules that express a rhNURR1 in a eukaryotic cell which has sufficient activity (ability to bind one or more of its binding partners or activate transcription from a NURR1 responsive promoter) to be useful in drug discovery.

The present invention further includes synthetic DNAs (sDNA) which encode the rhNURR1 wherein the nucleotide sequence of the sDNA differs from the nucleotide sequence of SEQ ID NO:1 but still encodes the rhNURR1 having the amino acid sequence as set forth in SEQ ID NO:2. For example, to express or enhance expression of the rhNURR1 in a particular cell type, it may be necessary to change the sequence comprising one or more of the codons encoding the rhNURR1 to a sequence which enables expression of the rhNURR1 in the particular cell type. Such changes include modifications for codon usage peculiar to a particular host or removing cryptic cleavage or regulatory sites which would interfere with expression of the rhNURR1 in a particular cell type. Therefore, the present invention discloses codon redundancies which may result in numerous DNA molecules expressing an identical protein. For purposes of this specification, a sequence bearing one or more replaced codons will be defined as a degenerate variation. Also included within the scope of this invention are mutations either in the DNA sequence or the translated protein that do not alter or do not substantially alter the ultimate physical or functional properties of the expressed protein (in general, these mutations are referred to as conservative mutations). For example, substitution of valine for leucine, arginine for lysine, or asparagine for glutamine may not cause a change in the functionality of the polypeptide.

It is known that DNA sequences encoding a peptide may be altered so as to code for a peptide that has properties that are different than those of the naturally occurring peptide. Methods for altering the DNA sequences include, but are not limited to, site-directed mutagenesis. Examples of altered properties include, but are not limited to, changes in the affinity of an enzyme for a substrate or a receptor for a ligand. In a particular embodiment, the present invention provides an isolated nucleic acid molecule comprising a sequence which encodes a mutated rhNURR1 comprising the sequence set forth in SEQ ID NO:2 with about 1 to 10 amino acid additions, deletions, or substitutions, wherein the mutated rhNURR1 binds at least a homologous or heterologous RXR, preferably a human RXR, or binds a nucleic acid comprising at least one NBRE binding site, or activates transcription from a natural or composite (synthetic) NURR1 responsive promoter.

Included in the present invention are nucleic acid sequences that hybridize to a nucleotide sequence in SEQ ID NO:1 under stringent conditions. By way of example, and not limitation, a procedure using conditions of high stringency is as follows. Prehybridization of filters containing nucleic acid molecules which might hybridize to a nucleotide sequence of SEQ ID NO:1 immobilized thereon is carried out for about two hours to overnight at about 65° C. in buffer composed of 6×SSC, 5× Denhardt's solution, and 100 μg/ml denatured salmon sperm DNA. Filters are hybridized for about 12 to 48 hrs at 65° C. in prehybridization mixture containing 100 μg/ml denatured salmon sperm DNA and 5-20×10⁶ counts-per-minute of ³²P-labeled nucleic acid probe comprising at least 10 contiguous nucleotides of the nucleotide sequence in SEQ ID NO:1. Washing of filters is done at 37° C. for about 1 hour in a solution containing 2×SSC, 0.1% SDS. This is followed by a wash in 0.1×SSC, 0.1% SDS at 50° C. for 45 minutes before detecting bound probe by autoradiography. Other procedures using conditions of high stringency would include either a hybridization step carried out in 5×SSC, 5× Denhardt's solution, 50% formamide at about 42° C. for about 12 to 48 hours or a washing step carried out in 0.2× SSPE, 0.2% SDS at about 65° C. for about 30 to 60 minutes. Reagents mentioned in the foregoing procedures for carrying out high stringency hybridization are well known in the art. Details of the composition of these reagents can be found in Sambrook et al., Molecular Cloning: A Laboratory Manual 2^{nd} Edition; Cold Spring Harbor Laboratory Press, Cold Spring Harbor, N.Y., (1989) or Sambrook and Russell, Molecular Cloning: A Laboratory Manual, 3rd Edition, Cold Spring Harbor Laboratory Press, Plainview, N.Y. (2001). In addition to the foregoing, other conditions of high stringency which may be used are also well known in the art.

In an another aspect of the present invention, a substantially purified form of an rhNURR1 which comprises a sequence of amino acids as disclosed in FIG. 2 (SEQ ID NO:2) is provided. Further provided are polypeptide fragments and/or mutants of the rhNURR1, preferably bioactive polypeptide fragments, which comprise at least a portion of the amino acid sequence set forth in SEQ ID NO: 2. By way of example, a polypeptide fragment of rhNURR1 can comprise the LBD from about amino acid 356 to about amino acid 598, DBD from about amino acid 261 to about amino acid 336, AF1 from about amino acid 1 to about amino acid 84, AF2 from about amino acid 583 to about amino acid 598, the H1 alpha helix domain from about amino acid 350 to about amino acid 398, and the H3 to H12 alpha helix domains from about amino acid 399 to about amino acid 398. These mutations or polypeptide fragments include, but are not limited to, amino acid substitutions, deletions, additions, amino terminal truncations, and carboxy-terminal truncations such that these mutations provide for proteins or protein fragments of diagnostic, therapeutic, or prophylactic use and are useful for screening assays for identifying analytes which interfere with the interaction of rhNURR1 and one or more homologous or heterologous binding partners or its ability to activate transcription from a NURR1 responsive promoter, such analytes being useful for treatment of an inflammatory disease such as inflammatory joint disease, ulcerative colitis, and thyroiditis, in particular arthritic diseases such as rheumatoid arthritis, osteoarthritis, psoriatic arthritis, and sarcoid arthritis, neurological disorders such as Parkinson's disease and bipolar disorders, or prostate diseases, including prostate cancer. The above polypeptides and fragments are useful for X-ray crystallography studies on the structure of particular domains of the rhNURR1 and their interactions with particular binding partners or analytes.

The rhNURR1 polypeptides of the present invention can be the “mature” protein or a fragment or portion thereof, any of which can be a part of a larger protein such as to provide a fusion or chimeric protein. It is often advantageous to include covalently linked to the amino acid sequence of the rhNURR1, an additional amino acid sequence which contains secretory or leader sequences, pro-sequences, sequences which aid in purification of the rhNURR1 such as multiple histidine residues (polyHis) or antibody-binding epitopes, or one or more additional amino acid sequences which confer stability to the rhNURR1 during recombinant production. Thus, rhNURR1 fusion proteins are provided which comprise all or part of the rhNURR1 linked at its amino or carboxyl terminus to proteins or polypeptides such as green fluorescent protein (GFP), c-myc epitope, alkaline phosphatase, protein A or G, glutathione S-transferase (GST), polyHis, peptide cleavage site, or antibody Fc region. In particular aspects of the fusion protein, the fusion protein can comprise the rhNURR1 LBD fused to the DBD of another protein, for example, the NURR1 LBD, AF1, AF2, or combinations thereof is fused to the DBD of the glucocorticoid receptor (GR) or the DBD of the yeast GAL4 protein. Any of the foregoing fusion constructs can be expressed in a cell line of interest and used to screen for modulators of the rhNURR1 disclosed herein. The present invention further provides an isolated nucleic acid molecule comprising a sequence which encodes a fusion rhNURR1 comprising all or a part of the sequence set forth in SEQ ID NO:2 or a mutated variant thereof in which the mutation comprises about 1-10 amino acid additions, deletions, or substitutions, operably linked to a nucleic acid encoding a second protein or fragment thereof including mutants thereof.

The present invention further provides vectors which comprise at least one of the nucleic acid molecules disclosed throughout this specification, preferably wherein the nucleic acid molecule is operably linked to a heterologous promoter. These vectors can comprise DNA or RNA. For most cloning purposes, DNA plasmid or viral expression vectors are preferred. Typical expression vectors include plasmids, modified viruses, bacteriophage, cosmids, yeast artificial chromosomes, and other forms of episomal or integrated DNA, any of which expresses the rhNURR1, polypeptide fragment thereof, or fusion protein comprising all or part of the rhNURR1 encoded therein. It is well within the purview of the skilled artisan to determine an appropriate vector for a particular gene transfer or other use. As used herein, the term “recombinant rhNURR1” is intended to include any variation of rhNURR1 disclosed herein which is expressed from a vector transfected into a eukaryote cell or transformed into a prokaryote cell. Transfected eukaryote cells and transformed prokaryote cells are referred to as recombinant host cells.

An expression vector containing DNA encoding a rhNURR1 or any one of the aforementioned variations thereof wherein the DNA is preferably operably linked to a heterologous promoter can be used for expression of the recombinant rhNURR1 in a recombinant host cell. Such recombinant host cells can be cultured under suitable conditions to produce recombinant rhNURR1 or a biologically equivalent form. Expression vectors include, but are not limited to, cloning vectors, modified cloning vectors, specifically designed plasmids, or specifically designed viruses. As used herein, the term “variants” includes both mutants and fragments of the rhNURR1.

Commercially available mammalian expression vectors which are suitable for recombinant rhNURR1 expression include, but are not limited to, pcDNA3.neo (Invitrogen, Carlsbad, Calif.), pcDNA3.1 (Invitrogen), pcDNA3.1/Myc-His (Invitrogen), pCI-neo (Promega, Madison, Wis.), pLITMUS28, pLITMUS29, pLITMUS38 and pLITMUS39 (New England Bioloabs, Beverly, Mass.), pcDNAI, pcDNAIamp (Invitrogen), pcDNA3 (Invitrogen), pMClneo (Stratagene, La Jolla, Calif.), pXT1 (Stratagene), pSG5 (Stratagene), EBO-pSV2-neo (ATCC 37593) pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

Also, a variety of bacterial expression vectors can be used to express recombinant rhNURR1 in bacterial cells. Commercially available bacterial expression vectors, which may be suitable for recombinant rhNURR1 expression include, but are not limited to, pCR2.1 (Invitrogen), pET11a (Novagen, Madison, Wis.), lambda gt11 (Invitrogen), pCR4Blunt-TOPO (Invitrogen), and pKK223-3 (Pharmacia).

In addition, a variety of fungal cell expression vectors may be used to express recombinant rhNURR1 in fungal cells. Commercially available fungal cell expression vectors which are suitable for recombinant rhNURR1 expression include, but are not limited to, pYES2 (Invitrogen) and Pichia expression vector (Invitrogen).

Also, a variety of insect cell expression vectors can be used to express recombinant rhNURR1 in insect cells. Commercially available insect cell expression vectors which can be suitable for recombinant expression of rhNURR1 include, but are not limited to, pBlueBacIII and pBlueBacHis2 (Invitrogen), and pAcG2T (Pharmingen).

Viral vectors which can be used for expression of recombinant rhNURR1 include, but are not limited to, adenovirus vectors, adeno-associated virus vectors, herpesvirus vectors, Sindbis virus vectors, Simliki forest virus vectors, pox virus vectors (such as vaccinia virus, fowl pox, canary pox, and the like), retrovirus vectors, and baculovirus vectors. Many of viral vectors are commercially available.

The present invention further provides recombinant host cells transformed or transfected with a vector comprising any one of the aforementioned nucleic acid molecules, particularly host cells transformed or transfected with a vector comprising any one of the aforementioned nucleic acid molecules wherein the nucleic acid molecule is operably linked to a promoter. Recombinant host cells include bacteria such as E. coli, fungal cells such as yeast, plant cells, mammalian cells including, but not limited to, cells of bovine, porcine, monkey, human, or rodent origin; and insect cells including, but not limited to, Drosophila and silkworm-derived cell lines. For instance, one insect expression system utilizes Spodoptera frugiperda (Sf21) insect cells (Invitrogen) in tandem with a baculovirus expression vector (pAcG2T, Pharmingen, San Diego, Calif.). Mammalian cells include primary cultures of chondrocytes, osteoblasts, synovial cells, and the like. Also, mammalian cell lines, which may be suitable, include but are not limited to, 22Rv1 cells (ATCC CRL-2505), LnCap cells (ATCC CRL-1740), hFOB 1.19 cells (ATCC CRL 11372), 7F2 cells (ATCC CRL 12557), SaOs-2 cells (ATCC HTB-85, MC3T3T3 cells (ATCC CRL-2593), HIG82 cells (ATCC CRL-1832), C-28/12 cells, T/C-28a2 cells, T/C-28a4, JEG3 cells, L cells L-M(TK-) (ATCC CCL-1.3), L cells L-M (ATCC CCL-1.2), 293 cells (ATCC CRL-1573), Raji cells (ATCC CCL-86), CV-1 cells (ATCC CCL-70), COS-1 cells (ATCC CRL-1650), COS-7 cells (ATCC CRL-1651), CHO-K1 cells (ATCC CCL-61), 3T3 cells (ATCC CCL-92), NIH/3T3 cells (ATCC CRL-1658), HeLa cells (ATCC CCL-2), C1271 cells (ATCC CRL-1616), BS-C-1 cells (ATCC CCL-26), MRC-5 cells (ATCC CCL-171), HEK293T cells (ATCC CRL-1573), ST2 cells (Riken Cell bank, Tokyo, Japan RCB0224), C3HIOT1/2 cells (JCRB0602, JCRB9080, JCRB003, or IFO50415), and CPAE cells (ATCC CCL-209). Such recombinant host cells can be cultured under suitable conditions to produce rhNURR1 or a biologically equivalent form.

As noted above, an expression vector containing DNA encoding rhNURR1 or any one of the aforementioned variations thereof can be used to express the rhNURR1 encoded therein in a recombinant host cell. Therefore, the present invention provides a process for expressing a rhNURR1 or any one of the aforementioned variations thereof in a recombinant host cell comprising introducing the vector comprising a nucleic acid which encodes the rhNURR1 into a suitable host cell and culturing the host cell under conditions which allow expression of the rhNURR1. In a further aspect, the rhNURR1 has an amino acid sequence substantially as set forth in SEQ ID NO:2 and binds at least one homologous or heterologous rhNURR1 binding partner such as RXR or nucleic acid comprising at least one NURR1 binding site, or activates transcription from a NURR1 responsive promoter such as the OPN promoter and the nucleic acid encoding the rhNURR1 or variation thereof is operably linked to a heterologous promoter which can be constitutive or inducible. Thus, the present invention further provides a cell comprising a nucleic acid encoding the rhNURR1 or variation thereof which has an amino acid sequence substantially as set forth in SEQ ID NO:2, which preferably binds at least one homologous or heterologous binding partner such as RXR or nucleic acid comprising at least one NURR1 binding site, or activates a NURR responsive promoter such as the OPN promoter, and wherein the nucleic acid encoding the rhNURR1 is operably linked to a heterologous promoter.

The nucleic acids of the present invention are preferably assembled into an expression cassette that comprises sequences which provide for efficient expression of the rhNURR1 or variant thereof as described previously encoded thereon in a mammalian cells such as a human cell. The cassette preferably contains the full-length cDNA encoding the rhNURR1 or a DNA encoding a fragment of the rhNURR1 with homologous or heterologous transcriptional and translational control sequences operably linked to the DNA. Such control sequences include at least a transcription promoter (constitutive or inducible) and transcription termination sequences and can further include other regulatory elements such as transcription enhancers, ribosome binding sequences, splice junction sequences, and the like. In most aspects, the promoter is a heterologous promoter; however, in particular aspects, the promoter can the natural rhNURR1 promoter for ectopic expression of the rhNURR1 in various host cells of non-rhesus monkey origin. In a particularly useful aspect, the promoter is the constitutive cytomegalovirus immediate early promoter with or without the intron A sequence (CMV) although those skilled in the art will recognize that any of a number of other known promoters such as the strong immunoglobulin promoter, Rous sarcoma virus long terminal repeat promoter, SV40 small or large T antigen promoter, or the like. A preferred transcriptional terminator is the bovine growth hormone terminator although other known transcriptional terminators such as SV40 termination sequences can also be used. The combination of an expression cassette comprising the rhNURR1 operably linked to the CMV promoter and the BGH terminator has been found to provide suitable expression of cDNA encoding the rhNURR1 in eukaryote cells.

Following expression of rhNURR1 or any one of the aforementioned variations of the rhNURR1 in a host cell, rhNURR1 or variant thereof can be recovered to provide rhNURR1 in a form capable of binding one or more homologous or heterologous NURR1 binding partners or activate transcription from a NURR1 responsive promoter. Several rhNURR1 purification procedures are available and suitable for use. The rhNURR1 can be purified from cell lysates and extracts by various combinations of, or individual application of salt fractionation, ion exchange chromatography, size exclusion chromatography, hydroxylapatite adsorption chromatography, or hydrophobic interaction chromatography. In addition, rhNURR1 can be separated from other cellular polypeptides by use of an immunoaffinity column made with monoclonal or polyclonal antibodies specific for rhNURR1 or a particular epitope thereof. Alternatively, in the case of fusion polypeptides comprising all or a portion of the rhNURR1 fused to a second polypeptide, purification can be achieved by affinity chromatography comprising a reagent specific for the second polypeptide such as an antibody or metal.

Methods for cloning, constructing expression vectors, producing recombinant host cells, including transiently or stably transfected eukaryote cells, protein isolation, and the like are well known in the art and can be found for example in Sambrook et al., Molecular Cloning: A Laboratory Manual 2nd Edition; Cold Spring Harbor Laboratory Press, Cold Spring Harbor, N.Y., (1989) or Sambrook and Russell, Molecular Cloning: A Laboratory Manual, 3rd Edition, Cold Spring Harbor Laboratory Press, Plainview, N.Y. (2001).

In accordance with yet another embodiment of the present invention, there are provided antibodies having specific affinity for the rhNURR1 or fragment or epitope thereof. The term “antibodies” is intended to be a generic term which includes polyclonal antibodies, monoclonal antibodies, Fab fragments, single V_{H} chain antibodies such as those derived from a library of camel or llama antibodies or camelized antibodies (Nuttall et al., Curr. Pharm. Biotechnol. 1: 253-263 (2000); Muyldermans, J. Biotechnol. 74: 277-302 (2001)), and recombinant antibodies. The term “recombinant antibodies” is intended to be a generic term which includes single polypeptide chains comprising the polypeptide sequence of a whole heavy chain antibody or only the amino terminal variable domain of the single heavy chain antibody (V_{H} chain polypeptides) and single polypeptide chains comprising the variable light chain domain (V_{L}) linked to the variable heavy chain domain (V_{H}) to provide a single recombinant polypeptide comprising the Fv region of the antibody molecule (scFv polypeptides)(See, Schmiedl et al., J. Immunol. Meth. 242: 101-114 (2000); Schultz et al., Cancer Res. 60: 6663-6669 (2000); Dübel et al., J. Immunol. Meth. 178: 201-209 (1995); and in U.S. Pat. No. 6,207,804 B1 to Huston et al.). Construction of recombinant single V_{H} chain or scFv polypeptides which are specific against an analyte can be obtained using currently available molecular techniques such as phage display (de Haard et al., J. Biol. Chem. 274: 18218-18230 (1999); Saviranta et al., Bioconjugate 9: 725-735 (1999); de Greeff et al., Infect. Inmun. 68: 3949-3955 (2000)) or polypeptide synthesis. In further aspects, the recombinant antibodies include modifications such as polypeptides having particular amino acid residues or ligands or labels such as horseradish peroxidase, alkaline phosphatase, fluors, and the like. Further still aspects include fusion polypeptides which comprise the above polypeptides fused to a second polypeptide such as a polypeptide comprising protein A or G.

The antibodies specific for rhNURR1 can be produced by methods known in the art. For example, polyclonal and monoclonal antibodies can be produced by methods well known in the art, as described, for example, in Harlow and Lane, Antibodies: A Laboratory Manual. Cold Spring Harbor Laboratory Press: Cold Spring Harbor, N.Y. (1988). rhNURR1 or fragments or epitopes thereof can be used as immunogens for generating such antibodies. Alternatively, synthetic peptides of particular regions of the rhNURR1 can be prepared (using commercially available synthesizers) and used as immunogens. Amino acid sequences can be analyzed by methods well known in the art to determine whether they encode hydrophobic or hydrophilic domains of the corresponding polypeptide. Altered antibodies such as chimeric, humanized, camelized, CDR-grafted, or bifunctional antibodies can also be produced by methods well known in the art. Such antibodies can also be produced by hybridoma, chemical synthesis or recombinant methods described, for example, in Sambrook et al., supra., and Harlow and Lane, supra. Both anti-peptide and anti-fusion protein antibodies can be used. (See, for example, Bahouth et al., Trends Pharmacol. Sci. 12: 338 (1991); Ausubel et al., Current Protocols in Molecular Biology (John Wiley and Sons, N.Y. (1989)).

Antibodies so produced can be used for the immunoaffinity or affinity chromatography purification of the rhNURR1 or rhNURR1/binding partner complexes. The above referenced anti-rhNURR1 antibodies can also be used to modulate the activity of the rhNURR1 in living animals, in humans, or in biological tissues isolated therefrom. Accordingly, contemplated herein are compositions comprising a carrier and an amount of an antibody having specificity for rhNURR1 effective to block naturally occurring rhNURR1 from binding to a NURR1 binding partner or activate transcription from a NURR1 responsive promoter.

Therefore, the nucleic acids encoding rhNURR1 or variants thereof, vectors containing same, host cells transformed with the nucleic acids or vectors which express the rhNURR1 or variants thereof, the expressed rhNURR1 or variants thereof, as well as antibodies specific for rhNURR1, can be used in in vivo or in vitro methods for screening a plurality of analytes to identify analytes which are modulators of the rhNURR1/binding partner interaction or transcription activation. These methods provide information regarding the function and activity of the rhNURR1 or variants thereof which can lead to the identification and design of molecule, compounds, or compositions capable of specific interactions with NURR1.

Therefore, the nucleic acids encoding rhNURR1 or variant thereof, vectors containing same, host cells transformed with the nucleic acids or vectors which express the rhNURR1 or variants thereof, the rhNURR1 and variants thereof, as well as antibodies specific for the rhNURR1, can be used in in vivo or in vitro methods for screening a plurality of analytes to identify analytes which are modulators of the rhNURR1/NURR1 binding partner (RXR, co-activator, other ligand, or nucleic acid comprising at least one NURR1 binding site) interaction. These methods provide information regarding the function and activity of the rhNURR1 and variants thereof which can lead to the identification and design of molecule, compounds, or compositions capable of specific interactions with human rhNURR1. In preferred aspects, the methods identify analytes that interfere with the binding of the rhNURR1 to a homologous or heterologous NURR1 binding partner involved in the various cellular signaling pathways. Such analytes are useful either alone or in combination with other compounds for treating inflammatory diseases such as osteoarthritis, bone disorders such as osteoporosis, prostate disorders, and neurological disorders such as Parkinson's Disease, schizophrenia, and bipolar disorders, and prostate disorder such as prostate cancer.

Accordingly, in a further aspect, the present invention provides methods (screening assays) for identifying analytes which modulate the binding of rhNURR1 to one or more homologous or heterologous binding partners. That is, screening methods for identifying candidates or test compounds or agents, for example, peptides, peptidomimetics, small molecules, or other drugs. Modulators can include, for example, agonists or antagonists. In a particularly preferred aspect, the present invention provides methods for identifying analytes which modulate binding of rhNURR1 to its protein or nucleic acid binding partner. In general, analytes which modulate binding of rhNURR1 to its binding partner either bind the rhNUUR1 or the binding partner and the binding modulates the binding of the NURR1 to its binding partner.

In preferred aspects, the screening methods disclosed herein are useful for identifying analytes which bind to the site or domain of rhNURR1 that is involved in binding to a particular NURR1 binding partner or to a site or domain on the NURR1 binding partner which is involved in binding to the rhNURR1. In either case, the screening methods identify analytes which interfere with the binding of rhNURR1 to a NURR1 binding partner. The interference in binding can be measured directly by identifying the rhNURR1/NURR1 binding partner complex or indirectly by monitoring a downstream cellular response to the interference in binding such as activation of a reporter gene responsive to the interference in binding.

Methods for identifying analytes which modulate (interfere with, inhibit, suppress, or stimulate) binding of rhNURR1 to one or more homologous or heterologous NURR1 binding partners (preferably, a heterologous NURR1 binding partner, most preferably a heterologous NURR1 binding partner of human origin) include (i) cell-based binding methods for identifying analytes which modulate rhNURR1 activity or inhibit or stimulate binding between rhNURR1 and at least one homologous or heterologous NURR1 binding partner; and (ii) cell-free binding methods for identifying analytes which inhibit binding between rhNURR1 protein and at least one homologous or heterologous NURR1 binding partner or nucleic acid comprising one or more NURR1 binding sites. Therefore, while the methods disclosed herein use the rhNURR1 and nucleic acids encoding the same, the NURR1 binding partner and nucleic acids encoding the same (where appropriate) are not limited to those obtained from the rhesus monkey but can include those polypeptides and nucleic acids encoding the same from other mammals such as humans.

In one aspect, the invention provides methods for screening a plurality of analytes for analytes which bind to or modulate the activity of rhNURR1 or polypeptide or biologically active portion thereof. In another aspect, the invention provides methods for screening a plurality of analytes for analytes which bind to or modulate the activity of NURR1 binding partner. The plurality of analytes can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12: 145 (1997)).

Examples of methods for synthesizing molecular libraries can be found in the art, for example, DeWitt et al., Proc. Natl. Acad. Sci. USA 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of analytes can be presented in solution (for example, Houghten, Biotech. 13: 412421 (1992)), or on beads (Lam, Nature 354: 82-84 (1991)), chips (Fodor Nature, 364: 555-556 (1993)), bacteria or spore (U.S. Pat. No. 5,223,409 to Ladner), plasmids (Cull et al., Proc Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. USA 87: 6378-6382 (1990); Felici J. Mol. Biol. 222:301-310(1991); and, U.S. Pat. No. 5,223,409 to Ladner).

In the various embodiments of the cell-based and cell-free methods disclosed herein, the NURR1 binding partner is preferably obtained from an organism selected from the group consisting of mouse, rat, dog, and human. More preferably, the NURR1 binding partner is of human origin. The NURR1 binding partner is preferably selected from the group consisting of RXR and nucleic acid comprising at least one NURR1 binding site.

The present invention provides a cell-based screening method for identifying analytes which modulate the rhNURR1's ability to stimulate transcription of a reporter gene operably linked to a promoter comprising an NURR1 binding site. The method entails co-transfecting into a mammalian cell a first vector which comprises a gene expression cassette comprising a nucleic acid encoding the rhNURR1 operably linked to a promoter and a second vector which comprises a gene expression cassette comprising a reporter gene encoding a detectable product operably linked to a promoter containing at least one NURR1 binding site. The promoter regulating expression of the rhNURR1 can be a heterologous promoter which is either inducible or a constitutive. The promoter regulating expression of the reporter gene can be the OPN, TH, or DAT promoter disclosed herein, or another promoter which is NURR1 responsive. Reporter genes which are useful in the cell-based assays of the present invention include, but are not limited to, the luciferase gene, green fluorescent protein, secreted alkaline phosphatase (SEAP), placental alkaline phosphatase (PLAP), β-galactosidase gene, β-lactamase gene, and β-glucoronidase gene.

In a further aspect of the above cell-based method, the first gene expression cassette comprises a nucleic acid encoding a fusion protein comprising the LBD of the rhNURR1 fused to the yeast GAL4 DBD and the second expression cassette comprises a nucleic acid encoding a reporter gene operably linked to a promoter comprising at least one copy of recognition site for the GAL4 DBD. The rhNURR1 LBD encoded by the nucleic acid comprises the amino acids from about position 356 to about position 598 and the GAL4 DBD comprises the amino acids from about position 1 to about position 147.

In a further still aspect of the above cell-based method, the first gene expression cassette comprises a nucleic acid encoding a fusion or chimeric protein comprising the LBD of the rhNURR1 fused to the glucocorticoid receptor (GR) DBD and the second expression cassette comprises a nucleic acid encoding a reporter gene operably linked to a promoter comprising at least one copy of a recognition site for the GR DBD. The rhNURR1 LBD encoded by the nucleic acid comprises the amino acids from about position 356 to about position 598 and the GR DBD comprises the amino acids from about position 1 to about position 505.

In a typical assay for antagonists or agonists, the cells transfected with the above vectors comprising any one of the above embodiments are incubated in a medium containing the analyte to be tested for antagonist or agonist activity. During the incubation period or a suitable period of time after commencing the incubation period, expression of the reporter gene is measured. In general, a positive control is also provided in which the transfected cells are incubated in a medium without the analyte and a negative control is provided in which the transfected cells are incubated in a medium containing a known antagonist. A decrease in expression of the reporter gene relative to the positive control indicates that the analyte is an antagonist of NURR1 activity whereas an increase or enhancement in expression of the reporter gene relative to the positive control indicates that the analyte is an agonist of NURR1 activity.

The present invention further provides a cell-based method for identifying analytes which are agonists or antagonists of the interaction between RXR and the rhNURR1. The method entails co-transfecting into a mammalian cell a first vector which comprises a gene expression cassette comprising a nucleic acid encoding the rhNURR1 operably linked to a promoter, a second vector which comprises a gene expression cassette comprising a reporter gene encoding a detectable product operably linked to a promoter containing at least one NURR1 binding site, and a third vector which comprises a gene expression cassette comprising a nucleic acid encoding the RXR operably linked to a heterologous promoter. The expression cassettes are transfected into a cell and the cell incubated in a medium comprising an analyte to be tested for agonist or antagonist activity. An analyte which is an agonist causes an increase in reporter gene expression whereas an analyte which is an antagonist causes a decrease in reporter gene expression. The promoters regulating expression of the rhNURR1 and the RXR can be heterologous promoters which are either inducible or a constitutive. The promoter regulating expression of the reporter gene is a promoter comprising one or more retinoid acid response elements, for example, promoter can be a composite promoter comprising the human retinoid acid receptor β1 (hRAR^{β}2) gene promoter (^{β}RE) upstream of a thymidine kinase promoter. Reporter genes which are useful in the cell-based assays of the present invention include, but are not limited to, the luciferase gene, green fluorescent protein, secreted alkaline phosphatase (SEAP), placental alkaline phosphatase (PLAP), β-galactosidase gene, β-lactamase gene, and β-glucoronidase gene. In particular embodiments, the RXR is provided by co-transfecting the first and second vectors into a cell which expresses high levels of endogenous RXR, for example, human Hep2 cells express high levels of endogenous RXR.

In a further aspect for identifying agonists and antagonists of the rhNURR1/RXR interaction, a two-hybrid assay is provided wherein the first gene expression cassette comprises a nucleic acid encoding a fusion protein comprising the LBD of the rhNURR1 fused to the GAL4 DBD operably linked to a promoter, the second expression cassette comprises a nucleic acid encoding a reporter gene operably linked to a promoter comprising at least one copy of a recognition site for the GAL4 DBD, and the third expression cassette comprises a nucleic acid encoding a fusion protein comprising the LBD of RXR fused to the VP16 activation domain of herpes simplex virus (HSV) operably linked to a promoter. Preferably, the RXR LBD comprises the human RXRα from about amino acid 198 to about amino acid 462. The expression cassettes are transfected into a cell and the cell incubated in a medium comprising an analyte to be tested for inhibiting heterodimerization between the rhNURR1 LBD and the RXR LBD. A two-hybrid assay using a fusion protein consisting of a NURR1 LBD fused to the GAL4 DBD and the RXR LBD fused to the VP 16 activation domain for defining the requirements for heterodimerization between NURR1 and RXR was described in Aarnisalo et al., J. Biol. Chem. 277: 35118-35123 (2002).

In a further still aspect of the two-hybrid assay, the first gene expression cassette comprises a nucleic acid encoding a fusion protein comprising the LBD of the rhNURR1 fused to the GR DBD operably linked to a promoter, the second expression cassette comprises a nucleic acid encoding a reporter gene operably linked to a promoter comprising at least one copy of a recognition site for the GR DBD, and the third expression cassette comprises a nucleic acid encoding a fusion protein comprising the LBD of RXR fused to the VP 16 activation domain of herpes simplex virus (HSV) operably linked to a promoter. Preferably, the RXR LBD comprises the human RXRα from about amino acid 198 to about amino acid 462.

In a typical assay for agonists or antagonists, the cells transfected with the above vectors comprising any one of the above embodiments are incubated in a medium containing the analyte to be tested for antagonist or agonist activity. During the incubation period or a suitable period of time after commencing the incubation period, expression of the reporter gene is measured. In general, a control is also provided in which the transfected cells are incubated in a medium without the analyte and a negative control is provided in which the transfected cells are incubated in a medium containing a known antagonist. A decrease in expression of the reporter gene relative to the positive control indicates that the analyte is an antagonist of the NURR1—RXR interaction whereas an increase in expression of the reporter gene relative to the control indicates that the analyte is an agonist of the NURR1-RXR interaction.

In a further still aspect, an rhNURR1 LBD assembly assay is provided which can identify analytes that either destabilize the rhNURR1 LBD or stabilize the NURR1 LBD. Castro et al. (J. Biol. Chem. 274: 37483-37490 (1999)) disclosed that NURR1 LBD was more active in 293 cells than in human chorine carcinoma JEG-3 cells. Wang et al. (Nature 423: 555-560 (2003)) used a NURR1 LBD assembly assay to show that stability of the NURR1 LBD correlated with cell-specific activity transcriptional activity observed by Castro et al. Their data suggests that the NURR1 LBD adopts a conformation in 293 cells that facilitates transcription activation whereas in JEG-3 cells, it adopts conformation that inhibits transcription activation. The rhNURR1 assembly assay comprises providing a first gene expression cassette comprising a nucleic acid encoding a fusion protein comprising the H1 alpha helix of the LBD fused to the GAL4 DBD, a second gene expression cassette comprising a fusion protein comprising the H3 to H 12 alpha helixes of the LBD fused to the strong activation domain VP 16 of herpes simplex virus, and a third gene expression cassette comprising a reporter gene operably linked to a promoter comprising one ore more GAL4 binding sites. When the gene expression cassettes are introduced into a cell that stabilizes the rhNURR1 LBD, the GAL4-H1 fusion protein binds the GAL4 binding site of the promoter and the H1 and the H3-H12 of the H3-H12-VP16 interact, which enables the VP16 to activate transcription of the reporter gene. In cells that do not stabilize the H1/H3-H12 interaction there is no transcription activation.

To identify analytes that destabilize the rhNURR1 LBD, the gene expression cassettes are transfected into a cell that stabilizes the NURR1 LBD, for example, 293 cells. The transfected cells are incubated in a medium containing an analyte and the cells are monitored for a decrease or loss of transcription of the reporter gene. A positive control includes an assay which uses an analyte known to destabilize the rhNURR1 LBD and a negative control includes an analyte which has no destabilizing effect.

To identify analytes which stabilize the rhNURR1 LBD, the gene expression cassettes are transfected into a cell that normally does not stabilize the NURR1 LBD, for example, JEG-3 cells. The transfected cells are incubated in a medium containing an analyte and the cells are monitored for an transcription of the reporter gene. A positive control includes an assay which uses an analyte known to stabilize the rhNURR1 LBD and a negative control includes an analyte which has no stabilizing effect.

The present invention further provides a method for identifying an analyte that is useful for inducing NURR1 expression in a prostate cancer cell. The method involves providing prostate cancer cells such as 22Rv1 cells or LNCap cells and setting up a multiplicity of cultures, each containing an aliquot of cells. To each culture is added an analyte to be tested for stimulating NURR1 expression in the cell. At various time points thereafter, the expression of NURR1 is measured. An increase in expression of NURR1 in the presence of the analyte compared to expression of the NURR1 in the absence of the analyte indicates that the analyte is useful for inducing expression of NURR1 in the prostate cancer. The above method is useful for identifying an analyte useful for treating prostate cancer in a mammal and can comprise a method for treating prostate cancer in a mammal, which comprises providing a prostate cancer cell; incubating the cell in a medium which includes an analyte; and measuring expression of NURR1 in the cell to identify an analyte that induces expression of NURR1 in the prostate cancer cell; and administering the analyte identified above to the mammal to treat the prostate cancer.

The present invention further provides a method for treating prostate cancer in a mammal wherein the treatment uses an analyte that had been identified as inducing NURR1 expression in prostate cancer cells from the mammal. The method involves obtaining prostate cancer cells from the mammal and providing a multiplicity of cultures of the cells wherein each of the cultures of the cells is incubated in a medium that includes an analyte to be tested for ability to induce NURR1 expression in the cells. NURR1 expression is measured in each of the cultures of cells to identify an analyte that induced the NURR1 expression in the particular cells of the mammal. The analyte which induces the NURR1 expression in the cells is administered to the mammal to treat the prostate cancer in the mammal.

In a further aspect of the above methods, the expression of the NURR1 is determined by measuring the amount of RNA encoding the NURR1 in the cell. In a further still aspect, the amount of RNA is measured by reverse-transcription polymerase chain reaction. In a further still aspect, the expression of the NURR1 is determined by measuring the amount of NURR1 polypeptide in the cell. In a further still aspect, the amount of NURR1 polypeptide is determined by using an antibody specific for the NURR1.

The present invention further provides a method for identifying an analyte that suppresses NURR1 expression in the cells of a joint from a mammal with osteoarthritis treating osteoarthritis in a mammal. The method involves inducing osteoarthritis in a rat and providing an analyte to the rat. Then, at various time points thereafter, the expression of NURR1 is measured in cells from the knees of the osteoarthritic rat. A decrease in expression of the NURR1 in the cells of the rat knees treated with the analyte indicates that the analyte is able to suppress expression of NURR1 in the cells of the joints of an osteoarthritic mammal. Analytes identified by the above method are useful for treating the osteoarthritis in the mammal.

The present invention further provides a method for identifying an analyte useful for treating osteoarthritis in a mammal, which comprises inducing osteoarthritis in a multiplicity of rats; providing to each of the rats an analyte to be tested for ability to suppress NURR1 expression in the cells of the joints of the rats; and, measuring expression of NURR1 in the cells of the knees of the osteoarthritic rat to identify an analyte which suppresses NURR1 expression in the cells; and, administering the analyte identified above to the mammal to treat the osteoarthritis.

In further aspects of the above methods, the osteoarthritis is induced in the rat by a chemical treatment, resistance training, or a surgical treatment. In a further still aspect, the NURR1 expression is determined in the cells obtained from an anterior cruciate ligament transection or ACL transection and medial meniscetomy of the knees from the osteoarthritic rat. In a further aspect of the method, the expression of the NURR1 is determined by measuring the amount of RNA encoding the NURR1 in the cells. In a further still aspect, the amount of RNA is measured by Taqman real-time quantitative polymerase chain reaction. In a further aspect of the method, the expression of the NURR1 is determined by measuring the amount of NURR1 polypeptide in the cells. In a further still aspect, the amount of NURR1 polypeptide is determined by using an antibody specific for the NURR1. In a further aspect, the cells are selected from the group consisting of chondrocytes and synovial cells.

Cell-free screening assays are provided for identifying analytes which are agonists or antagonists of rhNURR1 binding to a NURR1 binding partner. In one embodiment of a cell-free screening method, the method is a competition assay in which the ability of an analyte to effectively compete with the rhNURR1 for binding to a homologous or heterologous NURR1 binding partner such as RXR, a co-activator, ligand, or nucleic acid comprising at least one NURR1 binding site is determined. Binding of the analyte can be determined either directly or indirectly. Binding can be determined using labeled or unlabeled antibodies against rhNURR1, analyte, or NURR1 binding partner, the rhNURR1/NURR1 binding partner complex, labeled rhNURR1, and combinations thereof. Labels include, but are not limited to, radioactive isotopes, fluorescent dyes, enzymatic reporters such as alkaline phosphatase or horseradish peroxidase, donor-quencher fluorescent dyes, antibody recognition sites such as those provided by fusion polypeptides (for example, rhNURR1 fused to alkaline phosphatase or a myc antibody recognition sequence).

In a further aspect, the method includes contacting the rhNURR1 with the NURR1 binding partner to form a mixture, adding an analyte to the mixture, and determining the ability of the analyte to interfere with the binding of the rhNURR1 to a NURR1 binding partner, e.g., RXR or a nucleic acid comprising one or more NBRE binding sites. Immunoprecipitation is a particular type of cell-free method which is useful for identifying analytes which inhibit binding of rhNURR1 to a NURR1 binding partner.

In another aspect of a cell-free method, the rhNURR1 is contacted with an analyte and the ability of the analyte to inhibit or suppress subsequent binding of the rhNURR1 to a homologous or heterologous NURR1 binding partner is determined. Determining the ability of the analyte to inhibit or suppress binding of the rhNURR1 can be detected as discussed above for cell-based methods. Determining the ability of the rhNURR1 to bind to a NURR1 binding partner can also be accomplished using a technology such as real-time Biomolocular Interaction Analysis (BIA) (Sjolander and Urbaniczky, Anal. Chem. 63: 2338-2345 (1991) and Szabo et al., Curr. Opin. Struct. Biol. 5: 699-705 (1995)). As used herein, “BIA” is a technology for studying biospecific interactions in real time, without labeling any of the interactants (for example, BIACORE). Changes in the optical phenomenon surface plasmon resonance (SPR) can be used as an indication of real-time reactions between biological molecules.

In particular aspects of the above cell-free methods, it can be desirable to immobilize either rhNURR1 or NURR1 binding partner to facilitate separation of rhNURR1/NURR1 binding partner complexes from free rhNURR1 and NURR1 binding partner, as well as to accommodate automation of the method. Binding of analyte to rhNURR1, or interaction of a rhNURR1 with a homologous or heterologous NURR1 binding partner in the presence and absence of an analyte, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/rhNURR1 fusion proteins or glutathione-S-transferase/NURR1 binding partner can be adsorbed onto glutathione SEPHAROSE beads (Sigma Chemical, St. Louis, Mo.) or glutathione-derivatized microtitre plates, which are then combined with the analyte or the analyte and either the non-adsorbed NURR1 binding partner or rhNURR1, and the mixture incubated under conditions conducive to complex formation (for example, at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix and the level of rhNURR1 binding or activity determined using standard techniques.

Other methods for immobilizing proteins on matrices can also be used in the cell-free screening methods. For example, either rhNURR1 or a NURR1 binding partner can be immobilized using a conjugation of biotin and streptavidin. Biotinylated protein or target molecules can be prepared from biotin-NHS(N-hydroxy-succinimide) using techniques well known in the art (for example, the biotinylation kit available from Pierce Biotechnology, Rockford, Ill.), and immobilized in the wells of streptavidin-coated 96 well plates (Pierce Biotechnology). Alternatively, antibodies reactive with rhNURR1 or NURR1 binding partner but which do not interfere with binding of the rhNURR1 to RXR can be derivatized to the wells of the plate, and unbound NURR1 binding partner or rhNURR1 trapped in the wells by antibody conjugation. Methods for detecting such complexes, in addition to those described above for the GST-immobilized complexes, include immunodetection of complexes using antibodies reactive with the rhNURR1 or NURR1 binding partner, as well as enzyme-linked assays which rely on detecting an enzymatic activity associated with the rhNURR1 or NURR1 binding partner.

A further aspect of a cell-free binding method for identifying analytes which inhibit binding of rhNURR1 to NURR1 binding partner is a modification of the GST fusion pull-down assay. The GST fusion pull-down assay has been described in Sambrook and Russell, Molecular Cloning: A Laboratory Manual 3rd Edition. Cold Spring Harbor Laboratory Press: Plainview, N.Y. (2001). A GST pull-down kit is available from Pierce Biotechnology. In the modified GST fusion pull-down assay used herein, either the DNA encoding rhNURR1 or NURR1 binding partner is cloned in-frame with the GST of a pGEX vector (Amersham Pharmacia Bioscience, Piscataway, N.J.) and expressed as a GST fusion protein in the BL21 E. Coli. The expressed GST fusion protein is bound to an immobilized reduced glutathione support. Preferably, the immobilized glutathione support is provided as a column. Labeled NURR1 binding partner or rhNURR1 (labeled protein), respectively, is incubated with the bound GST fusion protein in the presence of an analyte. Afterwards, unbound labeled protein is removed and the GST fusion protein (bound or unbound to the labeled protein) is eluted from the support with imidazole. The amount of labeled protein bound to the eluted GST fusion protein is determined by detecting the label. If the analyte interferes with rhNURR1 binding to the NURR1 binding partner, there will be little or no detectable labeled protein eluted with the GST fusion protein compared to controls without the analyte. Conversely, if the analyte does not interfere with rhNURR1 binding to the NURR1 binding partner, the amount of labeled protein eluted with the GST fusion protein will be similar to the amount eluted in controls without the analyte.

It would be readily apparent to one of ordinary skill in the art that other cell-based or cell-free methods not disclosed herein can be adapted to use rhNURR1 to identify analytes which inhibit or suppress binding of rhNURR1 to a homologous or heterologous NURR1 binding partner. Therefore, the present invention is not limited to the methods disclosed herein but can include other assays provided the assay is adapted to use the rhNURR1 or nucleic acids disclosed herein.

In many drug screening programs which screen libraries of analytes (drug, compounds, natural extracts, compositions, and the like), high throughput assays are desirable because they maximize the number of analytes that can be surveyed in a given period of time. Assays which are performed in cell-free systems, such as can be derived with purified or semi-purified proteins, are often preferred as “primary” screens in that they can be generated to permit rapid development and relatively easy detection of an alteration in a molecular target which is mediated by an analyte. Moreover, the effects of cellular toxicity and/or bioavailability of the analyte can be generally ignored in the in vitro system, the assay instead being focused primarily on the effect of the analyte on the molecular target as may be manifest in an alteration of binding affinity with upstream or downstream elements. Accordingly, in an exemplary screening method of the present invention, an analyte is contacted with rhNURR1 or NURR1 binding partner. The rhNURR1 or NURR1 binding partner can be soluble, on a membrane surface, or immobilized on a solid substrate such as the surface of the wells of microtiter plate, bioassay chip, or the like. To the mixture of the analyte and the rhNURR1 or NURR1 binding partner is then added a composition containing NURR1 binding partner or rhNURR1 protein, respectively. Detection and quantification of complexes of rhNURR1 and RXR in the presence of the analyte provide a means for determining an analyte's efficacy at inhibiting or potentiating complex formation between rhNURR1 and NURR1 binding partner. The efficacy of the analyte can be assessed by generating dose response curves from data obtained using various concentrations of the test compound. Moreover, a control can also be performed to provide a baseline for comparison. For the control, isolated and purified rhNURR1 or NURR1 binding partner is added to a composition containing the NURR1 binding partner or rhNURR1 and the formation of a rhNURR1/NURR1 binding partner complex is quantified in the absence of the analyte.

In one aspect, high throughput screening methods involve providing a library containing a large number of potential NURR1 modulators (candidate compounds). Such “combinatorial chemical libraries” are then screened in one or more assays, to identify those library member's particular chemical species or subclasses that display a desired characteristic activity. The compounds thus identified can serve as conventional “lead compounds” or can themselves be used as potential NURR1 modulators.

Devices for the preparation of combinatorial libraries are commercially available (See, for example, 357 MPS, 390 MPS, Advanced Chem Tech, Louisville, Ky.; Symphony, Rainin, Woburn, Mass.; 433A Applied Biosystems, Foster City, Calif.; 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (See, for example, ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.).

Any of the assays described herein are amenable to high throughput screening. As described above, the analytes are preferably screened by the methods disclosed herein. High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (See, for example, Zymark Corp., Hopkinton, Mass.; Air Technical Industries, Mentor, Ohio; Beckman Instruments, Inc. Fullerton, Calif.; Precision Systems, Inc., Natick, Mass.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols. Thus, for example, Zymark Corp. provides technical bulletins describing screening systems for detecting the modulation of gene transcription, ligand binding, and the like.

The following examples are intended to promote a further understanding of the present invention.

### EXAMPLE 1

A clone comprising the cDNA encoding the rhNURR1 was prepared from total RNA isolated from rhesus monkey prostate tissue using a reverse transcription-polymerase chain reaction (RT-PCR) method.

Total RNA was isolated from rhesus monkey prostate tissue and used in a reverse transcription (RT) reaction that was primed with oligo (dT) and which used the SUPERSCRIPT First Strand Synthesis System available from Invitrogen, Carlsbad, Calif. After the RT reaction, 2 μL of the RT reaction product was amplified in a PCR using a high fidelity DNA polymerase mixture.

To PCR amplify DNA encoding rhNURR1, two rounds of amplification were performed. The first round used a PCR primer pair consisting of forward primer A, 5′-ATGCC TTGTG TTCAG-3′ (SEQ ID NO:7) and reverse primer A, 5′-TTAGA AAGGT AAAGT GTC-3′ (SEQ ID NO:8). The nucleotide sequences of the forward and reverse primers A were based on the nucleotide sequence of NURR1 from humans. Primary and secondary PCR cycling were as follows: 95° C. for three minutes; two cycles of 95° C. for 30 seconds, 53° C. for 30 seconds, and 72° C. for 1.5 minutes; 28 cycles of 95° C. for 30 seconds, 55° C. for 30 seconds, and 72° C. for 1.5 minutes; and, 72° C. extension time of seven minutes. The PCR reaction mixture was then stored at 4° C. until needed.

Two μL of the first round amplification was used in the second round of amplification using forward primer B, 5′-ATGCC TTGTG TTCAG GCGCA GTATG G-3′ (SEQ ID NO:9), and reverse primer B, 5′-TTAGA AAGGT AAAGT GTCCA GGAAA AGTTT GTC-3′ (SEQ ID NO:10). The second round B primers are extensions of the A primers. The PCR cycling conditions were the same as was used in the first round of amplification.

After the second round of amplification, the DNA fragments in the reaction mixture were resolved by agarose gel electrophoresis. DNA fragments migrating in the gel in the position expected for DNA having a length of about 1.8 kb (the expected size for a nucleic acid encoding a NURR1) were eluted from the gel and inserted into the plasmid pCR4Blunt-TOPO (Invitrogen, Carlsbad, Calif.) by blunt-end ligation to make plasmid pCR4-rhNURR1. The plasmid pCRBlunt-TOPO contains the ccdB gene in the multiple cloning site. The ccdB gene controls cell death in E. coli. DNA fragments inserted into the multiple cloning site disrupt expression of ccdB which enables the transformed cell to grow.

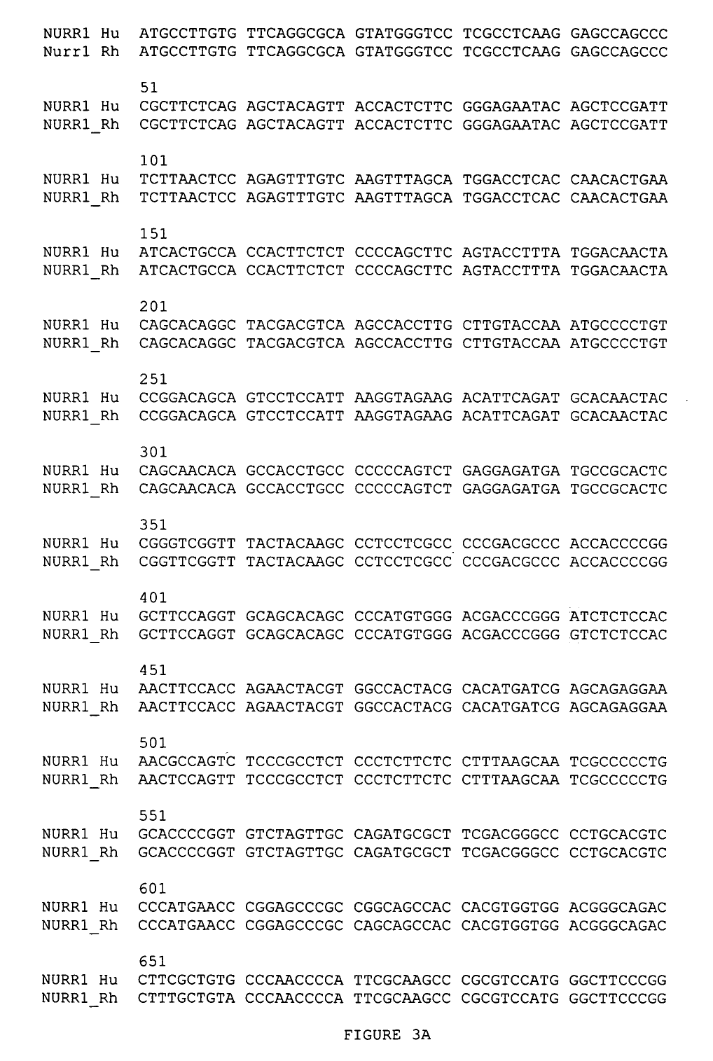

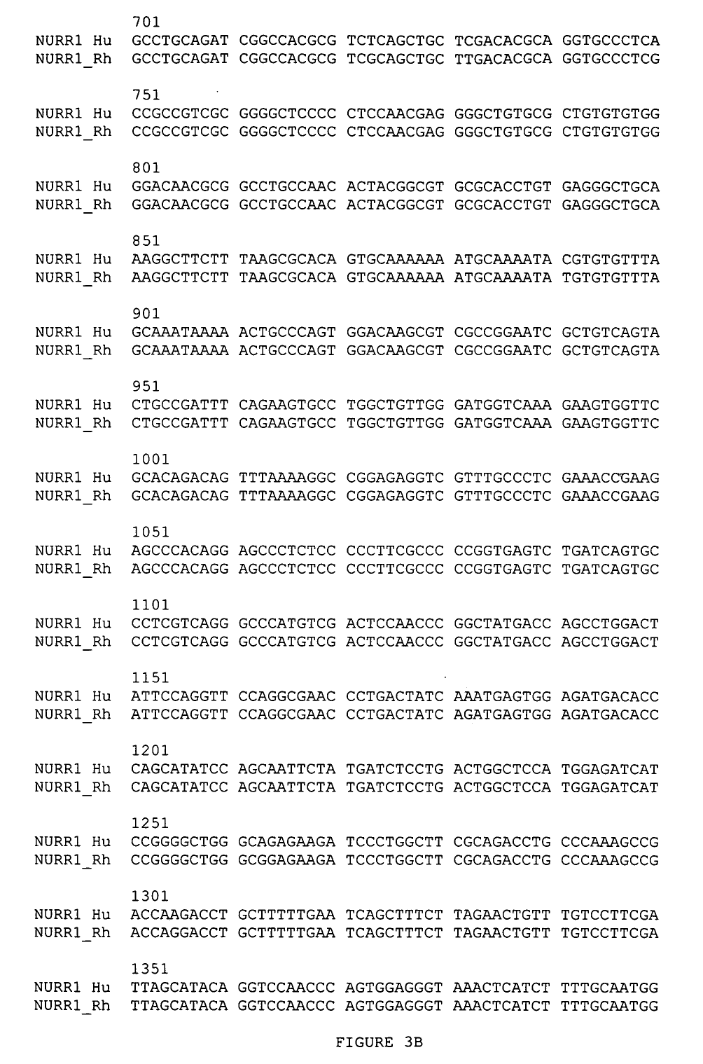

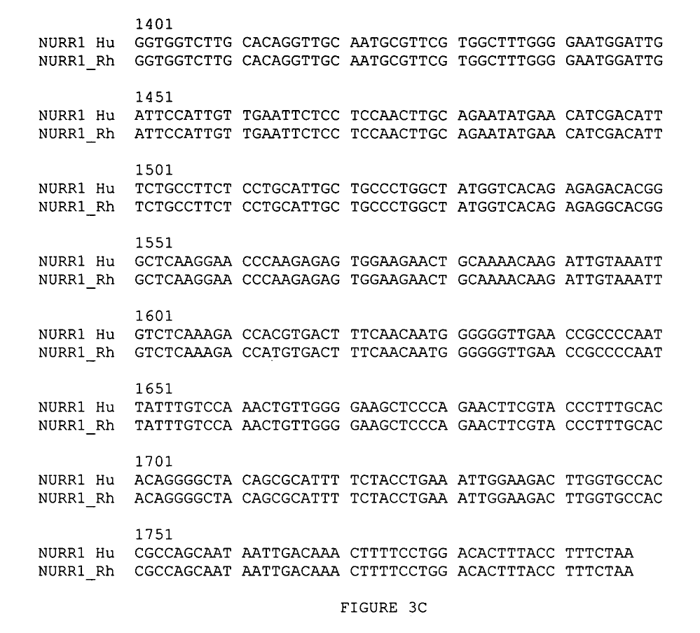

The plasmid was transformed into E. coli strain TOP 10F′ (Invitrogen Corp) and the transformants plated onto agar plates. DNA was extracted from several clones and sequenced. The nucleotide sequence of a cloned DNA fragment encoding rhNURR1 is shown in FIG. 1 and the deduced amino acid sequence is shown in FIG. 2. The amino acid and nucleotide sequences for the human NURR1 are available in Mages et al., Mol. Endocrinol. 8: 1583-1591 (1994) and WO9404675 to Kroczek and Mages. A comparison of the nucleotide sequence encoding the rhNURR1 to the nucleotide sequence encoding the human NURR1 is shown in FIGS. 3A, 3B, and 3C.

### EXAMPLE 2

A nucleic acid encoding a chimeric nuclear receptor comprising the rat glucocorticoid receptor (GR) DBD fused to the rhNURR1 LBD was constructed and the nucleic acid inserted downstream of the SV40 promoter in plasmid pSG5. The rat GR DBD consists of about amino acid 1 to about amino acid 505 and the rhNURR1 LBD consists of the amino acid sequence beginning from about amino acid 329 to about amino acid 598 (See FIG. 2). A nucleic acid encoding the rat GR DBD, which consisted of nucleotides 1 to 1583 (and which included the proximal 5′ untranslated sequences), was ligated to the nucleic encoding the rhNURR1 (nucleotides 329 to 1794). At the junction in the fusion protein between amino acid 505 of the rat GR DBD and amino acid 329 of the rhNURR1 LDB there are three extra amino acids, Alanine, Arginine, and Glutamic acid, which are encoded by the oligonucleotide 5′-GCTCGAGAA-3′ (SEQ ID NO:11) which includes an XhoI restriction enyme site.

The chimeric nuclear receptor was constructed by ligating the rat GRDBD encoding nucleotide fragment (nucleotides 1 to 1583) to the rhNURR1 LBD encoding nucleotide fragment (nucleotides 329 to 1794) and then inserting the ligated GRDBD/rhNURR1 LBD nucleotide fragments into the pSG5 plasmid expression vector between the EcoRI and BamHI sites.

A reporter gene expression cassette comprising the MMTV(2xGRE) promoter was constructed as described in Schmidt et al., Mol. Endocrinol. 6(10):1634-41 (1992).

### EXAMPLE 3

A routine transient transfection assay for identifying agonists of rhNURR1 activity is described. While the transfection uses the FuGENE6 method of Roche Applied Science, Indianapolis, Ind., other transfection methods can be used.

In general, the method consists of plating about 0.8 to 1×10⁴ transfected COS-1 or CHO cells per well of 96-well tissue culture plates which are UV and visible light transparent. The media that the cells are plated in is exponential growth medium which consists of phenol red-free DMEM containing 10% fetal bovine serum and 1× penicillin/streptomycin. Incubation conditions are 37° C. and 5% CO₂. The transfection is done in batch mode.

For a transfection, the cells are trypsinized and resuspended to about 8×10⁴ cells/mL in fresh medium. FuGENE6 is mixed with serum-free medium (for example, OPTIMEM, Invitrogen, Carlsbad, Calif.) at a ratio of 2 to 3. About 0.625 ng of DNA encoding the chimeric receptor of Example 2 and 0.625 ng DNA comprising the MMTV promoter operably linked to the luciferase gene (MMTV-LUC) are added to the FuGENE6 in serum-free medium and the mixture incubated at room temperature for about 20 minutes. The mixture is then added to one mL of the cell suspension and 100 μL aliquots plated to the wells of a 96-well tissue culture plate. Thus, a total ten wells contain cells, each containing about 8,000 cells. About 16 to 24 hours after transfection, for each well, about 1 uL of an analyte in a vehicle such as DMSO is added to the well. At least one well is reserved as a control and receives the vehicle alone. After 24 hours exposure to the analytes, the cells are assayed for luciferase activity. The cells are lysed by a Promega cell culture lysis buffer for approximately 30 min and then the luciferase activity in the extracts is assayed in the 96-well format liminometer (MICROBETA JET 1450, Perkin Elmer, Boston, Mass.). Results are calculated as fold induction of compound treated cells over control treated cells.

### EXAMPLE 4

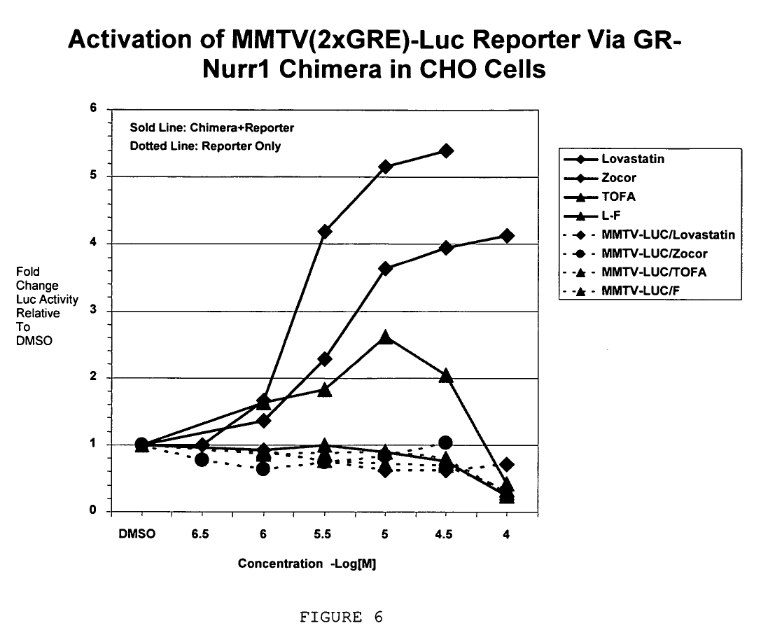

In this example, Lovastatin, Zocor, TOFA (5-(tetradecyloxy)-2-furoic acid) and SARM compound F were shown to be NURR1 agonists in an assay using CHO cells co-transfected with the GR/NURR1 chimera and reporter gene expression cassette of Example 2. The CHO cells were transfected as in Example 3. The assay was performed as in Example 3. The results are shown in FIG. 6 and show that ZOCOR and Lovistatin caused an increase in luciferase expression from the MMTV-LUC reporter plasmid compared to compound F and TOFA.

### EXAMPLE 5

The relative transactivation of GR/NURR1 chimera receptor in response to a panel of test analytes was determined.

To further verify the selective NURR1 LBD-compound interactions, the same compounds were added to cells that had been co-transfected with the MMTV-luciferase reporter gene expression cassette and a gene expression cassette comprising DNA encoding either the chimeric receptor GR/NURR1 or a chimeric GR/PPARα receptor under conditions similar to those in Example 5.

Expression vector 3GR/PPARα contained the chimeric GR/PPARα receptor in which DNA encoding the DNA binding domain of GR was fused to the ligand binding domain of mouse PPARα was inserted into plasmid pSG5 (See Boie et al., J. Biol. Chem. 268: 5530-55344 (1993)).

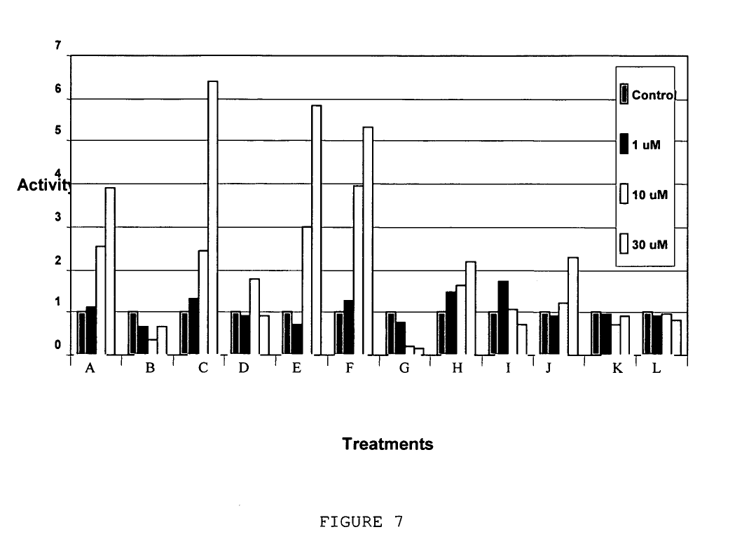

Then, aliquots of the transfected cells were treated with test analytes at 0, 1, 10, and 30 μM concentrations in DMSO. The test analytes included Bicalutamide, Lovastatin, ZOCOR, and various SARM compounds. The results are shown in FIG. 7 and are expressed as a relative activity in which the activity at each concentration that was obtained with the extracts from the cells cotransfected with GR/NURR1 was divided by the corresponding activity that was obtained from extracts from the cells cotransfected with GR/PPARα. Other chimeric receptors other than GR/PPARα can be used. This methods highlights the activity at each concentration of analyte that was selectively due to the analyte's interaction with the NURR1 LBD. As can be seen in FIG. 7, compounds A and C, Lovastatin (E), and ZOCOR (F) caused an increase in activity which suggests that they are NURR1 agonists.

### EXAMPLE 6

Relative transactivation of GR/NURR1 chimera receptor in response to a panel of test analytes was determined.

An MMTV-luciferase reporter gene expression cassette was cotransfected with a gene expression cassette comprising DNA encoding either the chimeric receptor GR/NURR1 or a chimeric receptor comprising GR/LXRβ under transfection conditions similar to those in Example 3.

Expression vector GR/LXRβ was prepared in a similar manner to the GR/PPARα chimeric receptor in Example 5. The nucleic acid encoding the rat GR amino terminus and DNA binding domain (DBD), which consisted of nucleotides 1 to 1583, was ligated to the nucleic acid encoding the LXRβ ligand binding domain (LBD)(NER1, nucleotides 707 to 2030 (See Shinar et al., Gene 147:273-276 (1994)). At the junction in the fusion protein between amino acid 1 to 505 of the rat GR and amino acid residues 155 to 461 of LXRβ (NER1). At the junction site there is an insertion of three extra amino acids, Alanine, Arginine, and Glutamic acid, which are encoded by the oligonucleotide 5′-GCTCGAGAA-3′.

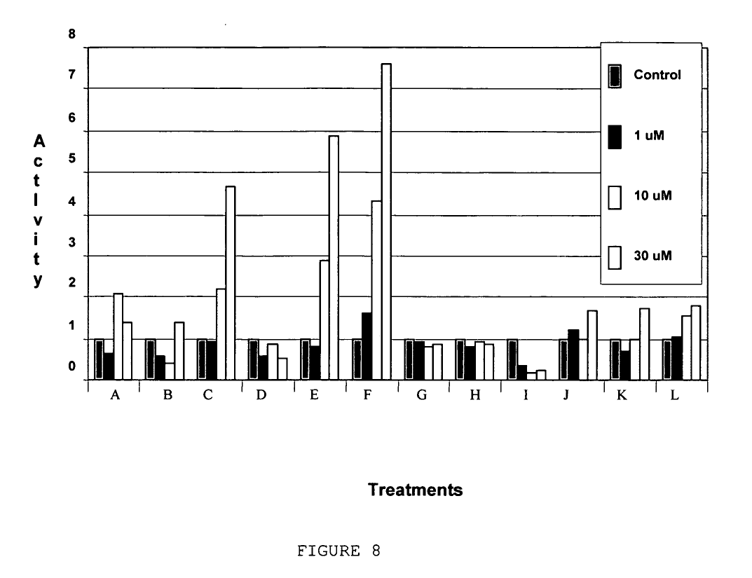

The next day, the transfected cells were washed and then refed with the appropriate medium containing 5% activated charcoal-stripped serum. Then, aliquots of the transfected cells were treated with test analytes at 0, 1, 10, and 30 μM concentrations in DMSO. The test analytes were Bicalutamide, Lovastatin, ZOCOR, and various SARM compounds. After 48 hours post-transfection, the cells were harvested and cell extracts prepared. The cell extracts were then tested for luciferase activity. The results are shown in FIG. 8 and are expressed as a relative activity in which the activity at each concentration that was obtained with cells transfected with the GR/NURR1 was divided by the corresponding activity that was obtained from cells cotransfected with the GR/LXRβ. This highlights the activity at each concentration of analyte that was selectively due to the analyte's interaction with the NURR1 LBD. As can be seen in FIG. 8, compound C, Lovastatin (E), and ZOCOR (F) caused an increase in activity which suggests that they are NURR1 agonists.

### EXAMPLE 7

The relative expression of NURR1 was evaluated in a variety of human and rat tissues as follows.

Panels of rat and human total RNA were purchased from several sources (Ambion (Austin, Tex.) and Clontech (Palo Alto, Calif.)). Bone tissue from rats was collected and processed as follows. Tibiae were rapidly dissected from animals and thoroughly cleaned to remove muscle and ligament as well as joint capsule, without causing undue damage to tibia plateau. Each tibia was then mounted in precision bone saw such that the medial side of the bone was oriented parallel with the holding clamp, while the head of the bone was oriented perpendicular to the clamp. Using the bone saw, thin slices were made from the end of each tibia. The thickness of each slice was approximately 960 μm from the tibeal plateau and contained articular cartilage (about 200 μm) and subchondral bone (about 760 μm). Each cartilage/bone slice was placed in saline and flushed with 23 gauge needle to remove bone marrow. Bone slices were pooled for each cut within a group. They were snapped frozen in liquid nitrogen and stored at −135° C. until processing.

Frozen tissue slices were immersed in liquid nitrogen and then crushed prior to addition to Trizol reagent (Life Technologies, Carlsbad, Calif.). Samples were homogenized using a Polytron (Brinkman Instruments, Westbury, N.Y.). Total RNA was isolated according to manufacturer's directions with minor modifications. Following the chloroform extraction step, RNA was re-extracted with acidic phenol:chloroform to remove proteoglycans. RNA was precipitated with isopropanol and washed with 75% ethanol. After pellet was air-dried, RNA was resuspended in molecular biology grade water (Eppendorf, Brinkman Instruments) and concentration of each sample was determined from A260 measurement. Agarose gel electrophoresis was used to confirm RNA integrity.

Primers and fluorogenic probes for TAQMAN polymerase chain reaction (PCR) real-time analysis were designed using PRIMER EXPRESS v. 1.0 (Applied Biosystems, Foster City, Calif.) and consisted of forward primer 5′-GGGTC CTCGC CTCAA GGA-3′ (SEQ ID NO:12), reverse primer 5-° CGGAG CTGTA TTCTC CCGAA-3′ (SEQ ID NO:13) and fluorogenic probe 5′-CCAGC CCCGC TTCTC AGAGC TACAG T-3′ (SEQ ID NO:14). All probes were synthesized by Applied Biosystems with the fluorescent reporter dye FAM (6-carboxy-fluorescein) attached to the 5′ end and the quencher dye TAMRA (6-carboxy-tetramethyl-rhodamine) attached to the 3′ end. Amplified products were designed to be between 70 to 110 bp. Commercially available human and rodent GAPDH (glyceraldehyde-3-phosphate dehydrogenase) primers and probe were purchased from Applied Biosystems and used for normalization. A PCR primer and probe set specific to Cyclophilin A was also used for normalization and consisted of forward primer 5′-CAAAT GCTGG ACCAA ACACA A-3′ (SEQ ID NO:15), reverse primer 5′-GCCAT CCAGC CACTC AGTCT-3′ (SEQ ID NO:16) and fluorogenic probe 5′-TGGTT CCCAG TTTTT TATCT GCACT GCC-3′ (SEQ ID NO:17).

All primer/probe sets were checked for efficiency of amplification using reverse transcribed rat bone total RNA. Standard curves were generated using serial dilutions of known quantities of total RNA in duplicate (e.g., input total RNA for PCR reaction at 50 ng RNA, 2 ng RNA, and 80 pg RNA).

RT reactions were carried out for each RNA sample in MICROAMP reaction tubes using TAQMAN reverse transcription reagents. Each reaction tube contained 250 ng of total RNA in a volume of 50 μL containing 1× TAQMAN RT buffer, 5.5 mM MgCl₂, 500 μM of each dNTP, 2.5 μM of oligo-d(T)16 primers, random hexamers, 0.4 U/μL RNase inhibitor, and 1.25 U/μL MULTISCRIBE Reverse Transcriptase. RT reaction was carried out at 25° C. for 10 minutes, 48° C. for 30 minutes, and 95° C. for 5 minutes. The RT reaction mixture was then placed at 4° C. for immediate use in PCR amplification or stored at −20° C. for later use.

Real-time PCR was performed in a MICROAMP Optical 96-well reaction plate. For each 50 μL reaction, 10 μL of RT product (50 ng total RNA), μM forward primer, μM reverse primer, μM probe and 1× Universal Master Mix (Applied Biosystems) were combined. Amplification conditions were 2 min at 50° C., 10 min at 95° C. followed by 40 cycles at 95° C. for 15 sec, 60° C. for 1 min.

All reactions were performed in ABI PRISM 7700 Sequence Detection System in duplicate using the SEQUENCE DETECTOR v 1.6 program.

The results, normalized to the expression levels of GPDH expression, are shown in FIGS. 4A and 4B. As shown in FIG. 4A, NURR1 is highly expressed in prostate tissue from normal men while its expression in tissue from the brain, heart, liver, lung, muscle, testis, and spleen is detectable but significantly less than the expression in prostate tissue. FIG. 4B shows that NURR1 is highly expressed in prostate tissue from normal rats with detectable to moderate expression in testes, brain, lung, and bone, particularly, in the articular cartilage and subchondral bone, the growth plate bone, and the metaphysis region of bone.

### EXAMPLE 8

NURR1 expression was found to be induced in osteoarthritic chondrocytes.

After inducing osteoarthritis by anterior cruciate ligament (ACL) transection or ACL transection and medial meniscetomy (MM)) surgery in groups of rats, the right and left tibiae from each of the groups of rats were rapidly dissected from the animals one, two, and four weeks following the surgery. A sham group was included. RNA was isolated from tissue slices obtained from the tibeal plateau containing both articular cartilage (about 200 μm) and subchondral bone (about 760 μm). Expression of NURR1 was then determined using TAQMAN real-time quantitative PCR. Procedures for RNA isolation, processing and quantitation were the same as described in Example 7. The relative levels of NURR1 expression were normalized to the expression levels of Cyclophilin A in the respective tissue.

As shown in FIG. 5, NURR1 expression was induced at an early stage in osteoarthritic chondrocytes taken from rats in which osteoarthritis had been induced.

### EXAMPLE 9

This example shows the relative expression of NURR1 in prostate and breast cancer cells in response to 50 nM DHT, Dexamethasone, 50 μM Biclutamide, 30 μM SARM compound A, 30 μM SARM compound J, and 10 μM SARM compound K in DMSO. Expression of NURR1 was determined using TAQMAN real-time quantitative PCR. Procedure for quantitating was the same as in Example 7.

FIG. 9 shows that NURR1 expression was induced in the prostate cancer cell lines 22Rv1 and LNCap treated with the SARM compound J. There was no significant expression of NURR1 in response to the other analytes. There was no significant expression of NURR1 in the breast cancer cell line MDA.

### EXAMPLE 10

This example describes a method for making polyclonal antibodies specific for rhNURR1.

Antibodies are generated in New Zealand white rabbits over a 10-week period. Purified rhNURR1 is emulsified by mixing with an equal volume of Freund's complete adjuvant and injected into three subcutaneous dorsal sites for a total of 0.1 mg rhNURR1 per immunization. A booster containing about 0.1 mg rhNURR1 emulsified in an equal volume of Freund's incomplete adjuvant is administered subcutaneously two weeks later. Animals are bled from the articular artery. The blood is allowed to clot and the serum collected by centrifugation. The serum is stored at −20° C.

For purification, rhNURR1 is immobilized on an activated support. Antisera is passed through the sera column and then washed. Specific antibodies are eluted via a pH gradient, collected, and stored in a borate buffer (0.125 M total borate) at −0.25 mg/mL. The anti-rhNURR1 antibody titers are determined using ELISA methodology with free rhNURR1 bound in solid phase (1 pg/well). Detection is obtained using biotinylated anti-rabbit IgG, HRP-SA conjugate, and ABTS.

### EXAMPLE 11

This example describes a method for making monoclonal antibodies specific for rhNURR1.

BALB/c mice are immunized with an initial injection of about 1 μg of purified rhNURR1 per mouse mixed 1:1 with Freund's complete adjuvant. After two weeks, a booster injection of about 1 μg of the antigen is injected into each mouse intravenously without adjuvant. Three days after the booster injection serum from each of the mice is checked for antibodies specific for the rhNURR1.

The spleens are removed from mice positive for antibodies specific for rhNURR1 and washed three times with serum-free DMEM and placed in a sterile Petri dish containing about 20 mL of DMEM containing 20% fetal bovine serum, 1 mM pyruvate, 100 units penicillin, and 100 units streptomycin. The cells are released by perfusion with a 23 gauge needle. Afterwards, the cells are pelleted by low-speed centrifugation and the cell pellet is resuspended in 5 mL 0.17 M ammonium chloride and placed on ice for several minutes. Then 5 mL of 20% bovine fetal serum is added and the cells pelleted by low-speed centrifugation. The cells are then resuspended in 10 mL DMEM and mixed with mid-log phase myeloma cells in serum-free DMEM to give a ratio of 3:1. The cell mixture is pelleted by low-speed centrifugation, the supernatant fraction removed, and the pellet allowed to stand for 5 minutes. Next, over a period of 1 minute, 1 mL of 50% polyethylene glycol (PEG) in 0.01 M HEPES, pH 8.1, at 37° C. is added. After 1 minute incubation at 37° C., 1 mL of DMEM is added for a period of another 1 minute, then a third addition of DMEM is added for a further period of 1 minute. Finally, 10 mL of DMEM is added over a period of 2 minutes. Afterwards, the cells are pelleted by low-speed centrifugation and the pellet resuspended in DMEM containing 20% fetal bovine serum, 0.016 mM thymidine, 0.1 hypoxanthine, 0.5 μM aminopterin, and 10% hybridoma cloning factor (HAT medium). The cells are then plated into 96-well plates.

After 3, 5, and 7 days, half the medium in the plates is removed and replaced with fresh HAT medium. After 11 days, the hybridoma cell supernatant is screened by an ELISA assay. In this assay, 96-well plates are coated with the rhNURR1. One hundred μL of supernatant from each well is added to a corresponding well on a screening plate and incubated for 1 hour at room temperature. After incubation, each well is washed three times with water and 100 μL of a horseradish peroxide conjugate of goat anti-mouse IgG (H+ L), A, M (1: 1,500 dilution) is added to each well and incubated for 1 hour at room temperature. Afterwards, the wells are washed three times with water and the substrate OPD/hydrogen peroxide is added and the reaction is allowed to proceed for about 15 minutes at room temperature. Then 100 μL of 1 M HCl is added to stop the reaction and the absorbance of the wells is measured at 490 nm. Cultures that have an absorbance greater than the control wells are removed to two cm2 culture dishes, with the addition of normal mouse spleen cells in HAT medium. After a further three days, the cultures are re-screened as above and those that are positive are cloned by limiting dilution. The cells in each two cm² culture dish are counted and the cell concentration adjusted to 1×10⁵ cells per mL. The cells are diluted in complete medium and normal mouse spleen cells are added. The cells are plated in 96-well plates for each dilution. After 10 days, the cells are screened for growth. The growth positive wells are screened for antibody production; those testing positive are expanded to 2 cm2 cultures and provided with normal mouse spleen cells. This cloning procedure is repeated until stable antibody producing hybridomas are obtained. The stable hybridomas are progressively expanded to larger culture dishes to provide stocks of the cells.

Production of ascites fluid is performed by injecting intraperitoneally 0.5 mL of pristane into female mice to prime the mice for ascites production. After 10 to 60 days, 4.5×10⁶ cells are injected intraperitoneally into each mouse and ascites fluid is harvested between 7 and 14 days later.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. An isolated nucleic acid molecule comprising a nucleotide sequence encoding a rhesus monkey NURR1 polypeptide or fragment thereof having an amino acid sequence of SEQ ID NO:2.

2. An isolated nucleic acid comprising a nucleotide sequence of SEQ ID NO:1.

3. An isolated polypeptide comprising an amino acid sequence of SEQ ID NO:2.

4. An antibody which binds a polypeptide comprising an amino acid sequence of SEQ ID NO:2.

5. A vector comprising a nucleic acid encoding a rhesus monkey NURR1 polypeptide or fragment thereof having an amino acid sequence of SEQ ID NO:2.

6. A cell comprising a nucleic acid encoding a rhesus monkey NURR1 polypeptide or fragment thereof having an amino acid sequence of SEQ ID NO:2 wherein the nucleic acid is operably linked to a heterologous promoter.

7. A method for producing a rhesus monkey NURR1 polypeptide comprising:
(a) providing a nucleic acid encoding the NURR1 polypeptide operably linked to a heterologous promoter;
(b) introducing the nucleic acid into a cell to produce a recombinant cell; and
(c) culturing the recombinant cell under conditions which allow expression of the nucleic acid encoding the rhNURR1 polypeptide to produce the NURR1 polypeptide.

8. A method for identifying an analyte that binds a rhesus monkey NURR1 (rhNURR1) polypeptide, which comprises:
(a) providing the rhNURR1 polypeptide and the analyte in a mixture; and
(b) determining whether the analyte binds the rhNURR1 polypeptide in the mixture.

9. A method for identifying an analyte that interferes with the binding of a rhesus monkey NURR1 (rhNURR1) polypeptide to an rhNURR1 binding partner, which comprises:
(a) providing the rhNURR1 polypeptide, the rhNURR1 binding partner, and the analyte in a mixture; and
(b) determining whether the analyte interferes with the binding of the rhNURR1 polypeptide to the rhNURR1 binding partner in the mixture.

10. A method for identifying an analyte that modulates activity of a rhesus monkey NURR1 (rhNURR1), which comprises:
(a) providing a recombinant cell that expresses the rhNURR1;
(b) incubating the recombinant cell in a medium which includes the analyte; and
(c) measuring activity of the rhNURR1, wherein a change in the activity of the rhNURR1 in the presence of the analyte indicates the analyte modulates the activity of the rhNURR1.

11. A method for identifying an analyte that modulates activity of a rhesus monkey NURR1 (rhNURR1), which comprises:
(a) providing a recombinant cell which expresses the rhNURR1 and an assayable reporter gene product wherein the reporter gene is operably linked to a promoter responsive to the rhNURR1;
(b) incubating the recombinant cell in a medium which includes an analyte; and
(c) measuring the expression of the reporter gene product wherein an increase or decrease of expression of the reporter gene compared to expression in a recombinant cell in the absence of the analyte identifies the analyte that modulates activity of the rhNURR1.

12. The method of claim 1 wherein the recombinant cell further expresses a retinoid X receptor (RXR).

13. The method of claim 12 wherein expression of the RXR is ectopic.

14. The method of claim 12 wherein expression of the RXR is endogenous.

15. A method for identifying an analyte that modulates heterodimerization between a rhesus monkey NURR1 (rhNURR1) and a retinoid X receptor (RXR), which comprises:
(a) providing a recombinant cell which expresses the rhNURR1 and an assayable reporter gene product wherein the reporter gene is operably linked to a promoter responsive to the rhNURR1/RXR heterodimer;
(b) incubating the recombinant cell in a medium which includes an analyte; and
(c) measuring the expression of the reporter gene product wherein an increase or decrease of expression of the reporter gene compared to expression in a recombinant cell in the absence of the analyte identifies the analyte that modulates the heterodimerization between the rhNURR1 and the RXR.

16. The method of claim 15 wherein expression of the RXR is ectopic.

17. The method of claim 15 wherein expression of the RXR is endogenous.

18. A method for identifying an analyte that stabilizes the ligand binding domain (LBD) of a rhesus monkey NUR1 (rhNURR1), which comprises:
(a) providing a cell that does not stabilize the LBD of the rhNURR1;
(b) transfecting the cell with a first gene expression cassette encoding a first fusion protein comprising the H1 alpha helix domain of the LBD of the rhNURR1 fused to a heterologous DNA binding domain (DBD), a second gene expression cassette encoding a second fusion protein comprising the remainder of the LBD of the rhNURR1 fused to a heterologous transcription activation domain, and a third gene expression cassette encoding an assayable reporter gene product wherein the reporter gene is operably linked to a promoter which binds the heterologous DBD and is activated by the heterologous activation domain to produce a recombinant cell;
(c) incubating the recombinant cell in a medium which includes an analyte; and
(d) measuring the expression of the reporter gene product wherein expression of the reporter gene product indicates the analyte stabilizes the LBD of the rhNURR1.

19. A method for identifying an analyte that destabilizes the ligand binding domain (LBD) of a rhesus monkey NURR1 (rhNURR1), which comprises:
(a) providing a cell that stabilizes the LBD of the rhNURR1;
(b) transfecting the cell with a first gene expression cassette encoding a first fusion protein comprising the H1 alpha helix domain of the LBD of the rhNURR1 fused to a heterologous DNA binding domain (DBD), a second gene expression cassette encoding a second fusion protein comprising the remainder of the LBD of the rhNURR1 fused to a heterologous transcription activation domain, and a third gene expression cassette encoding an assayable reporter gene product wherein the reporter gene is operably linked to a promoter which binds the heterologous DBD and is activated by the heterologous activation domain to produce a recombinant cell;
(c) incubating the recombinant cell in a medium which includes an analyte; and
(d) measuring the expression of the reporter gene product wherein no expression of the reporter gene product indicates the analyte destabilizes the LBD of the rhNURR1.

20. A method for identifying an analyte useful for treating prostate cancer in a mammal, which comprises:
(a) providing a prostate cancer cell;
(b) incubating the cell in a medium which includes an analyte; and
(c) measuring expression of NURR1 in the cell, wherein an increase in expression of NURR1 in the prostate cancer cell in the presence of the analyte compared to expression of the NURR1 in the prostate cancer cell in the absence of the analyte indicates that the analyte is useful for treating the prostate cancer in the mammal.

21. A method for treating prostate cancer in a mammal, which comprises:
(a) providing a prostate cancer cell;
(b) incubating the cell in a medium which includes an analyte; and
(c) measuring expression of NURR1 in the cell to identify an analyte that induces expression of NURR1 in the prostate cancer cell; and
(d) administering the analyte identified in step (c) to the mammal to treat the prostate cancer.

22. A method for identifying an analyte for treating osteoarthritis in a mammal, which comprises:
(a) inducing osteoarthritis in a mouse;
(b) providing the analyte to the mouse; and
(c) measuring expression of NURR1 in the cells of the knees of the mouse, wherein a decrease in expression of the NURR1 in the cells of the knees mouse treated with the analyte indicates that the analyte is useful for treating the prostate cancer in the mammal.

23. A method for treating osteoarthritis in a mammal, which comprises:
(a) inducing osteoarthritis in a rat;
(b) providing an analyte to the rat; and
(c) measuring expression of NURR1 in the cells of the knees of the rat to identify an analyte that suppresses expression of the NURR1 in the cells; and
(d) administering the analyte identified in step (c) to treat the osteoarthritis in the mammal.
